(19) United States
(12) Patent Application Publication
(10) Pub. No.: US 2025/0207106 A1
(43) Pub. Date: Jun. 26, 2025

(54) COMPOSITION AND METHODS FOR ADENO-ASSOCIATED VIRUS PRODUCING STABLE HUMAN EMBRYONIC KIDNEY-293 CELL LINES

(71) Applicant: GENENTECH, INC., South San Francisco, CA (US)

(72) Inventors: Chi Kin Domingos Ng, South San Francisco, CA (US); Amy Shen, South San Francisco, CA (US); Gavin Christian Bamard, South San Francisco, CA (US); Min Lu, South San Francisco, CA (US)

(73) Assignee: GENENTECH, INC., South San Francisco, CA (US)

(21) Appl. No.: 18/990,358

(22) Filed: Dec. 20, 2024

Related U.S. Application Data
(60) Provisional application No. 63/667,323, filed on Jul. 3, 2024.
(60) Provisional application No. 63/612,667, filed on Dec. 20, 2023.

(51) Int. Cl. C12N 7/00; C12N 5/071

(52) U.S. Cl. CPC C12N 7/00; C12N 5/0686; C12N 2510/02; C12N 2750/00022; C12N 2750/14152

(57) ABSTRACT

The presently disclosed subject matter relates to compositions and methods for the generation of stable human embryonic kidney-293 (HEK-293) cell lines for adeno-associated virus (AAV) production.

### CROSS-REFERENCE TO RELATED APPLICATIONS

This application claims the benefit under 35 U.S.C. 119 (e) to U.S. Provisional Patent Application No. 63/612,667, filed on Dec. 20, 2023, and U.S. Provisional Patent Application No. 63/667,323, filed on Jul. 3, 2024, the entire contents of which are incorporated by reference herein.

### TECHNICAL FIELD

The presently disclosed subject matter relates to compositions and methods for the generation of stable human embryonic kidney-293 (HEK-293) cell lines for adeno-associated virus (AAV) production.

### BACKGROUND

AAV vectors offers many advantages in the gene therapy context over other viral vectors. The non-integrating, non-pathogenic properties of AAV vectors, coupled with abilities to transduce dividing as well as non-dividing cells with potential for long-term stable transgene expression make them the vehicle of choice for clinical applications. In addition, AAV vectors can be engineered to alter their tissue tropism and to evade host immunity, thereby enabling more efficient transduction of the intended target tissue and successful establishment of transgene expression.

Given the advantages of AAV vectors, there has been an increasing demand to develop stable mammalian host cells for their production. There are, however, many challenges to achieving such manufacturing-grade virus production. For example, AAV production by transient transfection of production cell lines with plasmids containing AAV genes, along with genes associated with adenoviral helper, capsid, and replicase functions, is fraught with poor scalability and reproducibility, leading to high manufacturing costs. Toxicity of viral genes also contribute to poor manufacturing efficiency thereby increasing costs. Moreover, conventional strategies for production cell line development involve random integration of nucleotide sequences, which is not only a rare event, but also may result in a range of undesirable gene expression and cell growth phenotypes, e.g., unstable cell growth. Heterogeneity resulting from such random integration processes often require time-consuming and labor-intensive screening of clones to isolate cell lines demonstrating a desirable level and quality of viral titer. To add to these drawbacks, the use of adherent cells for virus production has limited options for scale-up, making manufacturing prohibitively expensive. Moreover, many manufacturing platforms using adherent cells show poor performance in serum-free medium, whereas use of serum can lead to contamination of the viral particles, which increases downstream processing costs. Accordingly, there is a need in the art for cost-effective compositions and methods that enable more efficient, GMP-compliant viral vector production.

### SUMMARY OF THE INVENTION

The presently disclosed subject matter relates, in part, to stable mammalian host cells suitable for AAV production. In certain embodiments, the stable mammalian host cell comprises a first exogenous nucleic acid sequence integrated at a targeted locus of the genome of the stable mammalian host cell (TI host cell), and a second exogenous nucleic acid sequence that is integrated at least once in the genome of the mammalian cell. In this embodiment, the first exogenous nucleic acid sequence comprises two recombination recognition sequences (RRSs) encoding one or more of adeno-associated virus (AAV) replicase (REP), AAV capsid (CAP), adenovirus (Ad) E2A, Ad E4, Ad E4orf6, Ad E4orf6/7, Ad E2-DNA Binding Protein (E2DBP), or Ad virus-associated (Ad VA) gene products, e.g., proteins and/or RNA, and a first selection marker. Also in this embodiment, the second exogenous nucleic acid sequence comprises inverted terminal repeats (ITRs) flanking coding sequences for a polypeptide of interest (POI), one or more of AAV REP, AAV CAP, Ad E2A, Ad E4, Ad E4orf6, Ad E4orf6/7, Ad E2DBP, or Ad VA and a second selection marker.

In certain embodiments, the present disclosure provides a stable mammalian host cell where the first exogenous nucleic acid sequence encodes AAV REP, Ad E2A, Ad E4, and Ad VA. In certain embodiments, the second exogenous nucleic acid sequence encodes AAV CAP.

In certain embodiments, the mammalian cell comprises one to ten additional exogenous nucleic acids comprising ITRs flanking coding sequences for one or more of AAV REP, AAV CAP, Ad E2A, Ad E4, Ad E4orf6, Ad E4orf6/7, Ad E2DBP, or Ad VA, and a selection marker, where the one to ten additional exogenous nucleic acids are each integrated at least once in the genome of the mammalian cell. In certain embodiments, the one to ten additional exogenous nucleic acids each comprise distinct selection markers. In certain embodiments, two or more of the one to ten additional exogenous nucleic acids comprise the same selection marker. In certain embodiments, one or more of the one to ten additional exogenous nucleic acids further comprise a coding sequence for a polypeptide of interest (POI) within the flanking ITRs.

In certain embodiments, the stable mammalian host cell is a stable human cell (e.g., a HEK293 cell line).

In certain embodiments, integration of the first exogenous nucleic acid sequence can be promoted by an exogenous nuclease. In certain embodiments, the exogenous nuclease can be selected from the group consisting of a zinc finger nuclease (ZFN), a ZFN dimer, a transcription activator-like effector nuclease (TALEN), a TAL effector domain fusion protein, an RNA-guided DNA endonuclease, an engineered meganuclease, and a clustered regularly interspaced short palindromic repeats (CRISPR)-associated (Cas) endonuclease.

In certain embodiments, one or more proteins encoded by the first exogenous nucleic acid sequence is inducibly expressed. In certain embodiments, one or more of the AAV proteins encoded by the first exogenous nucleic acid sequence is inducibly expressed. In certain embodiments, one or more proteins encoded by the second exogenous nucleic acid sequence is inducibly expressed. In certain embodiments, one or more of the AAV proteins encoded by the second exogenous nucleic acid sequence is inducibly expressed.

In certain embodiments, one or more of the proteins encoded by the first exogenous nucleic acid sequence is inducibly expressed and one or more of the proteins encoded by the second exogenous nucleic acid sequence is constitutively expressed. In certain embodiments, one or more of the proteins encoded by the first exogenous nucleic acid sequence is inducibly expressed and one or more of the proteins encoded by the second exogenous nucleic acid sequence is inducibly expressed. In certain embodiments, one or more of the proteins encoded by the first exogenous nucleic acid sequence is constitutively expressed and one or more of the proteins encoded by the second exogenous nucleic acid sequence is constitutively expressed. In certain embodiments, one or more of the proteins encoded by first exogenous nucleic acid is constitutively expressed and one or more of the proteins encoded by the second exogenous nucleic acid is inducibly expressed. In yet other embodiments, a plurality of proteins encoded by the first and/or second exogenous nucleic acid sequences are inducibly expressed.

In certain embodiments, the inducibly expressed proteins are induced by the same inducing agent.

The presently disclosed subject matter also provides methods of producing recombinant AAV (rAAV) of interest. In certain embodiments, the method comprises: A) providing a stable mammalian host cell comprising a first exogenous nucleotide sequence integrated at a targeted locus of the genome of the mammalian host cell, wherein the first exogenous nucleotide sequence comprises two recombination recognition sequences (RRSs) flanking a first selection marker; B) introducing into the stable mammalian host cell, a second exogenous nucleic acid sequence, where the second exogenous nucleic acid sequence comprises two RRSs matching the two RRSs of the first exogenous nucleotide sequence and flanking a sequence encoding one or more of AAV REP, AAV CAP, Ad E2A, Ad E4, Ad E4orf6, Ad E4orf6/7, Ad E2DBP, or Ad VA proteins and a second selection marker; C) introducing a recombinase or a nucleic acid encoding a recombinase, wherein the recombinase recognizes the RRSs; D) simultaneously with B) and C), or sequentially after B) and C), introducing, via transposon-mediated genomic integration, a third exogenous nucleic acid sequence encoding one or more of AAV REP, AAV CAP, Ad E2A, Ad E4, Ad E4orf6, Ad E4orf6/7, Ad E2-DNA Binding Protein (E2DBP), Ad VA gene products, e.g., proteins and/or RNA, or a polypeptide of interest, and a third selection marker into the genome of the stable mammalian host cell; E) selecting for those mammalian host cells that stably express the second and third selection markers; and F) culturing the selected mammalian host cell under conditions sufficient to produce recombinant AAV. In certain embodiments, such methods may further comprise recovering the AAV of interest from the cell culture.

In certain embodiments, the second exogenous nucleic acid sequence encodes AAV REP, Ad E2A, Ad E4, and Ad VA. In certain embodiments, the third exogenous nucleic acid sequence encodes AAV CAP. In certain embodiments, the mammalian host cell is a human cell (e.g., HEK293).

In certain embodiments, the method comprises: A) providing a stable mammalian host cell comprising a first exogenous nucleotide sequence integrated at a targeted locus of the genome of the mammalian host cell, wherein the first exogenous nucleotide sequence comprises a first and a second RRS flanking at least one first selection marker, and a third RRS located between the first and the second RRS, and where all the RRSs are heterospecific; B) introducing into the cell provided in a) a first vector comprising two RRSs matching the first and the third RRS on the at least one integrated exogenous nucleotide sequence and flanking a sequence encoding one or more of AAV REP, AAV CAP, Ad E2A, Ad E4, Ad E4orf6, Ad E4orf6/7, Ad E2DBP, or Ad VA proteins and a selection marker; c) introducing into the cell provided in a) a second vector comprising two RRSs matching the second and the third RRS on the at least one integrated exogenous nucleotide sequence and flanking a sequence encoding one or more of AAV REP, AAV CAP, Ad E2A, Ad E4, Ad E4orf6, Ad E4orf6/7, Ad E2DBP, or Ad VA proteins and a selection marker; D) introducing two or more recombinases, or one or more nucleic acids encoding two or more recombinases, wherein the two or more recombinases recognize the RRSs; and E) simultaneously with B), C), and D), or sequentially after B), C), and D), introducing, via transposon-mediated genomic integration, an additional exogenous nucleic acid sequence encoding one or more of AAV REP, AAV CAP, Ad E2A, Ad E4, Ad E4orf6, Ad E4orf6/7, Ad E2-DNA Binding Protein (E2DBP), Ad VA gene products, e.g., proteins and/or RNA, or a polypeptide of interest, and a selection marker into the genome of the stable mammalian host cell; F) selecting for those mammalian host cells that stably express one or more of the selection markers; and G) culturing the selected mammalian host cell under conditions sufficient to produce recombinant AAV. In certain embodiments, such methods may further comprise recovering the AAV of interest from the cell culture.

In certain embodiments, a targeted integration of the first exogenous nucleotide sequence is promoted by an exogenous nuclease. In certain embodiments, the exogenous nuclease can be selected from the group consisting of a zinc finger nuclease (ZFN), a ZFN dimer, a transcription activator-like effector nuclease (TALEN), a TAL effector domain fusion protein, an RNA-guided DNA endonuclease, an engineered meganuclease, and a clustered regularly interspaced short palindromic repeats (CRISPR)-associated (Cas) endonuclease.

In certain embodiments, expression of the one or more proteins is controlled by a regulatable promoter. In certain embodiments, the regulatable promoter is selected from the group consisting of SV40 and CMV promoters.

In certain embodiments, the one or more of the proteins encoded by the first exogenous nucleotide sequence integrated at a targeted locus of the genome of the stable mammalian host cell is inducibly expressed. In certain embodiments, the one or more of the proteins encoded by the second exogenous nucleotide sequence integrated at a targeted locus of the genome of the stable mammalian host cell is inducibly expressed. In certain embodiments, one or more of the proteins encoded by the transposon-mediated genomically integrated third exogenous nucleic acid is inducibly expressed. In certain embodiments, one or more of the proteins encoded by the transposon-mediated genomically integrated third exogenous nucleic acid is inducibly expressed. In certain embodiments, one or more of the proteins encoded by the first exogenous nucleotide sequence integrated at a targeted locus of the genome of the stable mammalian host cell is inducibly expressed and one or more of the proteins encoded by the transposon-mediated genomically integrated third exogenous nucleic acid is constitutively expressed. In certain embodiments, one or more of the proteins encoded by the first exogenous nucleotide sequence integrated at a targeted locus of the genome of the stable mammalian host cell is inducibly expressed and one or more of the proteins encoded by the transposon-mediated genomically integrated third exogenous nucleic acid is inducibly expressed. In certain embodiments, one or more of the proteins encoded by the first exogenous nucleotide sequence integrated at a targeted locus of the genome of the stable mammalian host cell is constitutively expressed and one or more of the proteins encoded by the transposon-mediated genomically integrated third exogenous nucleic acid is constitutively expressed. In certain embodiments, one or more of the proteins encoded by first exogenous nucleotide sequence integrated at a targeted locus of the genome of the stable mammalian host cell is constitutively expressed and one or more of the proteins encoded by the transposon-mediated genomically integrated third exogenous nucleic is inducibly expressed.

In certain embodiments, the stable mammalian cells selected for expressing the third selection marker comprise one to ten copies of the transposon-mediated genomically integrated third exogenous nucleic acid sequence.

In certain embodiments, a plurality of proteins encoded by the first and/or second exogenous nucleic acid are inducibly expressed. In certain embodiments, the inducibly expressed proteins are induced by the same inducing agent.

### BRIEF DESCRIPTION OF THE DRAWINGS

FIGS. 1A-1B depict exemplary strategies for generating integrated stable mammalian host cell pools and isolated single cell clones (SCC) using targeted integration (TI). FIG. 1A illustrates sequential transfection approach for TI. FIG. 2A illustrates simultaneous transfection approach for TI.

FIG. 2 shows an exemplary knock-in landing cassette encoding a GFP gene and a hygromycin-thymidine kinase (HYTK) selection marker, flanked by two LoxP sites, L3 and 2L, an additional LoxP site, Loxfas, and an frt3 site.

FIG. 3 shows a comparison of AAV titers obtained using HEK293 TI (Target Integration) host cells and wild type (wt) HEK293 host cells.

FIGS. 4A-4E depict exemplary plasmids for generating AAV producing integrated stable host cells. FIG. 4A shows a Front TI plasmid comprising a Tet inducible promoter for driving expression of REP gene, an expression cassette of transactivator (rTA) and a downstream SV40 promoter. FIG. 4B shows a Back TI plasmid comprising a puromycin-N-acetyltransferase (PAC) gene followed by a Tet inducible promoter for driving the expression of helper genes. FIG. 4C shows a PiggyBac Plasmid #1 comprising a Tet-inducible promoter for driving expression of a Cap gene, followed by the gene of interest (GOI) sequence flanked by ITR sequences and a selection marker (SM), for example, a blasticidin (BSD), a zeocin (ZEO), or a hygromycin (HYGRO) resistance gene. FIG. 4D shows a PiggyBac Plasmid #2 comprising a Tet-inducible promoter for driving expression of a REP gene, followed by the zeocin resistance gene (ZEO).

FIG. 4E shows a PiggyBac Plasmid #3 comprising a Tet-inducible promoter for driving expression of helper genes, followed by the zeocin resistance gene (ZEO).

FIGS. 5A-5C depict exemplary combinations of CAP and GOI-encoding PiggyBac (PB) plasmids that can be transfected into a TI packaging host to make producer cell lines. FIG. 5A depicts a PB plasmid comprising a Tet-inducible promoter driving the expression of the CAP gene, followed by a GOI expression cassette flanked by two inverted terminal repeats (ITR) and a fused replication and transcription activator/blasticidin (rTA-Bsd) selection marker. FIG. 5B shows a PB plasmid comprising a Tet-inducible promoter driving the expression of the CAP gene, followed by two GOI expression cassettes, each flanked by two inverted terminal repeats (ITR) and a fused replication and transcription activator/Zeocin (rTA-Zeo) selection marker. FIG. 5C shows a PB plasmid comprising a Tet-inducible promoter driving the expression of the CAP gene, followed by two GOI expression cassettes, each flanked by two inverted terminal repeats (ITR) and a fused replication and transcription activator/hygromycin (rTA-Hyg) selection marker.

FIG. 6A-6F illustrates a method for screening TI pre-packaging clones comprising first and second rounds of screening. FIG. 6A is a schematic of the first and second rounds of screening. FIG. 6B illustrates the first round of screening, which is based on a replication assay. FIG. 6C shows the results of the replication assay versus stable TI/P pool data. FIG. 6D shows the results of screening 190 clones using the replication assay. FIG. 6E depicts the second round of screening, which is based on a transient transfection assay. FIG. 6F shows the results of screening 36 clones (identified in the replication assay) using the transient transfection assay.

FIG. 7 depicts to generating TI/P producer pools from clones identified using the replication and transient transfection assays.

FIG. 8 illustrates Cap/GOI plasmids with various combinations of double and triple selection markers used for TI/P producer pool production.

FIGS. 9A-9D show the results of protein expression, integrated copy number and rAAV titer analysis in the TI/P producer pools. FIG. 9A shows the results of GFP expression in cells transfected with double and triple selection plasmids. FIG. 9B shows the integrated copy numbers of Cap/GOI in cells transfected with double and triple selection plasmids. FIG. 9C shows the rAAV titers in cells transfected with double and triple selection plasmids. FIG. 9D shows the rAAV titers using a different TI pool.

FIGS. 10A-10D show exemplary plasmids and method for generating a pre-packaging pool containing REP and Helper genes using 2-plasmid RMCE strategy. FIG. 10A shows an exemplary method for generating producer clones. FIG. 10B depicts exemplary plasmids for generating AAV producing integrated stable host cells. FIG. 10C shows the full/empty ratio of rAAV purified from crude lysates generated from TI/P producer clones by mass photometry (MP) using the method and plasmids shown in FIGS. 10A and 10B. FIG. 10D shows the full/empty ratio of rAAV purified from crude lysates generated from triple plasmid transient transfection.

### DETAILED DESCRIPTION

The presently disclosed subject matter relates to TI host cells suitable for AAV production where the TI host cells are also subjected to transposon-mediated genomic integration of one or more exogenous nucleic acids, as well as methods of producing and using said combined transposon-mediated genomic integration and TI host cells. In certain embodiments, the host cells, genetic constructs (e.g., vectors), compositions, and methods described herein can be employed in the development and/or use of a combined transposon-mediated genomic integration and TI host cell. In certain embodiments, the preset disclosure is directed to an integration (“TIP”) strategy for manufacturing stable mammalian cells for AAV production where the TI and transposon-mediated genomic integration steps are combined. In certain embodiments, the present disclosure is directed to a sequential integration (“TI/P”) strategy for manufacturing stable mammalian host cells for AAV production where the TI and transposon-mediated genomic integration steps are performed sequentially.

For purposes of clarity of disclosure and not by way of limitation, the detailed description is divided into the following subsections:
1. Definitions
2. AAV vector production
3. Integration Sites
4. Exogenous Nucleotide Sequences
5. Methods of Targeted Integration and Transposon-Mediated Genomic Integration
6. Methods of Producing Recombinant AAV
7. Examples

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the presently disclosed subject matter. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As used herein, the term “selection marker” can be a gene that allows cells carrying the gene to be specifically selected for or against, in the presence of a corresponding selection agent. For example, but not by way of limitation, a selection marker can allow the host cell transformed with the selection marker gene to be positively selected for in the presence of the gene; a non-transformed host cell would not be capable of growing or surviving under the selective conditions. Selection markers can be positive, negative, or bi-functional. Positive selection markers can allow selection for cells carrying the marker, whereas negative selection markers can allow cells carrying the marker to be selectively eliminated. A selection marker can confer resistance to a drug or compensate for a metabolic or catabolic defect in the host cell. In prokaryotic cells, amongst others, genes conferring resistance against ampicillin, tetracycline, kanamycin, or chloramphenicol can be used. Resistance genes useful as selection markers in eukaryotic cells include, but are not limited to, genes for aminoglycoside phosphotransferase (APH) (e.g., hygromycin phosphotransferase (HYG), neomycin and G418 APH), dihydrofolate reductase (DHFR), thymidine kinase (TK), glutamine synthetase (GS), asparagine synthetase, tryptophan synthetase (indole), histidinol dehydrogenase (histidinol D), and genes encoding resistance to puromycin, blasticidin, bleomycin, phleomycin, chloramphenicol, Zeocin, and mycophenolic acid. Further marker genes are described in WO 92/08796 and WO 94/28143.

Beyond facilitating a selection in the presence of a corresponding selection agent, a selection marker can alternatively provide a gene encoding a molecule normally not present in the cell, e.g., green fluorescent protein (GFP), enhanced GFP (eGFP), synthetic GFP, yellow fluorescent protein (YFP), enhanced YFP (eYFP), cyan fluorescent protein (CFP), mPlum, mCherry, tdTomato, mStrawberry, J-red, DsRed-monomer, mOrange, mKO, mCitrine, Venus, YPet, Emerald, CyPet, mCFPm, Cerulean, and T-Sapphire. Cells harboring such a gene can be distinguished from cells not harboring this gene, e.g., by the detection of the fluorescence emitted by the encoded polypeptide.

As used herein, the term “operably linked” refers to a juxtaposition of two or more components, wherein the components are in a relationship permitting them to function in their intended manner. For example, a promoter and/or an enhancer is operably linked to a coding sequence if the promoter and/or enhancer acts to modulate the transcription of the coding sequence. In certain embodiments, DNA sequences that are “operably linked” are contiguous and adjacent on a single chromosome. In certain embodiments, e.g., when it is necessary to join two protein encoding regions, such as a secretory leader and a polypeptide, the sequences are contiguous, adjacent, and in the same reading frame. In certain embodiments, an operably linked promoter is located upstream of the coding sequence and can be adjacent to it. In certain embodiments, e.g., with respect to enhancer sequences modulating the expression of a coding sequence, the two components can be operably linked although not adjacent. An enhancer is operably linked to a coding sequence if the enhancer increases transcription of the coding sequence. Operably linked enhancers can be located upstream, within, or downstream of coding sequences and can be located a considerable distance from the promoter of the coding sequence. Operable linkage can be accomplished by recombinant methods known in the art, e.g., using PCR methodology and/or by ligation at convenient restriction sites. If convenient restriction sites do not exist, then synthetic oligonucleotide adaptors or linkers can be used in accord with conventional practice. An internal ribosomal entry site (IRES) is operably linked to an open reading frame (ORF) if it allows initiation of translation of the ORF at an internal location in a 5′ end-independent manner.

As used herein, the term “expression” refers to transcription and/or translation. In certain embodiments, the level of transcription of a desired product can be determined based on the amount of corresponding mRNA that is present. For example, mRNA transcribed from a gene of interest can be quantitated by PCR or by Northern hybridization. In certain embodiments, protein encoded by a gene of interest can be quantitated by various methods, e.g., by ELISA, by assaying for the biological activity of the protein, or by employing assays that are independent of such activity, such as Western blotting or radioimmunoassay, using antibodies that recognize and bind to the protein.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell but may contain mutations. Mutant progeny that have the same function or biological activity as screened or selected for in the originally transformed cell are included herein.

The term “nucleic acid molecule” or “polynucleotide” includes any compound and/or substance that comprises a polymer of nucleotides. Each nucleotide is composed of a base, specifically a purine- or pyrimidine base (i.e., cytosine (C), guanine (G), adenine (A), thymine (T) or uracil (U)), a sugar (i.e., deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule may be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of a proteins required for AAV production by the stable mammalian host cell. Such DNA (e.g., cDNA) or RNA (e.g., mRNA) vectors, can be unmodified or modified. For example, mRNA can be chemically modified to enhance the stability of the RNA vector and/or expression of the encoded molecule so that mRNA can be injected into a subject to generate the POI in vivo (see e.g., Stadler et al, Nature Medicine 2017, published online 12 Jun. 2017, doi: 10.1038/nm.4356 or EP 2 101 823 B1).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extra chromosomally or at a chromosomal location that is different from its natural chromosomal location.

As used herein, the term “vector” refers to a nucleic acid molecule capable of propagating another nucleic acid to which it is linked. The term includes the vector as a self-replicating nucleic acid structure as well as the vector incorporated into the genome of a host cell into which it has been introduced. In certain embodiments, vectors direct the expression of nucleic acids to which they are operatively linked. Such vectors are referred to herein as “expression vectors.”

As used herein, the term “homologous sequences” refers to sequences that share a significant sequence similarity as determined by an alignment of the sequences. For example, two sequences can be about 50%, 60%, 70%, 80%, 90%, 95%, 99%, or 99.9% homologous. The alignment is carried out by algorithms and computer programs including, but not limited to, BLAST, FASTA, and HMME, which compares sequences and calculates the statistical significance of matches based on factors such as sequence length, sequence identify and similarity, and the presence and length of sequence mismatches and gaps. Homologous sequences can refer to both DNA and protein sequences.

As used herein, the term “flanking” refers to that a first nucleotide sequence is located at either a 5′ or 3′ end, or both ends of a second nucleotide sequence. The flanking nucleotide sequence can be adjacent to or at a defined distance from the second nucleotide sequence. There is no specific limit of the length of a flanking nucleotide sequence. For example, a flanking sequence can be a few base pairs or a few thousand base pairs. In certain embodiments, the length of a flanking nucleotide sequence can be about at least 15 base pairs, at least 20 base pairs, at least 30 base pairs, at least 40 base pairs, at least 50 base pairs, at least 75 base pairs, at least 100 base pairs, at least 150 base pairs, at least 200 base pairs, at least 300 base pairs, at least 400 base pairs, at least 500 base pairs, at least 1,000 base pairs, at least 1,500 base pairs, at least 2,000 base pairs, at least 3,000 base pairs, at least 4,000 base pairs, at least 5,000 base pairs, at least 6,000 base pairs, at least 7,000 base pairs, at least 8,000 base pairs, at least 9,000 base pairs, at least 10,000 base pairs.

As used herein, the term “exogenous” indicates that a nucleotide sequence does not originate from a host cell and is introduced into a host cell by traditional DNA delivery methods, e.g., by transfection, electroporation, or transformation methods. The term “endogenous” refers to that a nucleotide sequence originates from a host cell. An “exogenous” nucleotide sequence can have an “endogenous” counterpart that is identical in base compositions, but where the “exogenous” sequence is introduced into the host cell, e.g., via recombinant DNA technology.

### 2. AAV Vector Production

The adeno-associated virus (AAV) is a nonenveloped, capsid virus that encompasses a 4.7 kb single-stranded DNA, which encodes four replication proteins (Rep; Rep78, Rep68, Rep52, Rep40), three capsid proteins (VP; VP1/VP2/VP3) and an assembly-activating protein (AAP). The coding region is flanked by a pair of inverted terminal repeats (ITRs), which serve as primers for genome replication. Wild type AAV is generally replication-defective and requires the presence of helper factors from a helper virus, e.g., as an adenovirus (Ad).

Ad helper factors, E1A, E1B55K, E2A, E4orf6, E4orf6/7, E2DBP and the VA RNA have been identified as relevant to AAV replication. An exemplary AAV production method involves the multi-plasmid transfection of HEK293 cells (a human cell line expressing the Ad El gene), which results in efficient rAAV production without the need for separate infection with a helper virus. This method of transfection, however, is inefficient, has poor scalability and reproducibility and is also associated with high manufacturing costs. In addition to these disadvantages, the property of HEK293 cells to constitutively express ElA activates AAV promoters leading to Rep and helper protein induced cytotoxicity, which poses major challenges to creating stable producer and packaging cell lines. As an alternative, regulated expression system can be used to express toxic proteins relevant to AAV production, for example, but not by way of limitation, where the sequence(s) encoding the potentially toxic protein(s) are under the control of an inducible promoter.

### 3. Integration Sites

The presently disclosed subject matter provides a mammalian host cell suitable for targeted integration (TI) and/or transposon-mediated genomic integration of nucleotide sequences required for AAV production. In certain embodiments, host cells of the present disclosure comprise a locus at which a first exogenous nucleic acid sequence is integrated by targeted integration. In certain embodiments, host cells of the present disclosure comprise a second exogenous nucleic acid sequence integrated at least once in the genome of the host cell. In certain embodiments, host cells of the present disclosure comprise one to ten additional exogenous nucleic acids sequences integrated at least once in the genome of the host cell. In certain embodiments integration of the second exogenous nucleic acid sequence is mediated using a transposon.

In certain embodiments, the mammalian host cell is a human host cell, for example a human embryonic kidney 293 (HEK293) cell or variants thereof.

In certain embodiments, the mammalian host cell is said to be stable for AVV production if the viral titers produced by the cell are maintained at a certain level, increases, or decreases less than 20%, over 10, 20, 30, 50, 100, 200, or 300 generations. In certain embodiments, mammalian host cell is said to be stable for AVV production if the viral production is maintained without any selection.

An “integration site” comprises a nucleic acid sequence within a host cell genome into which a first exogenous nucleotide sequence is inserted. In certain embodiments, an integration site is between two adjacent nucleotides on the host cell genome. In certain embodiments, an integration site includes a stretch of nucleotides between any of which an exogenous nucleotide sequence can be inserted. In certain embodiments, the integration site is located within a specific locus of the genome of the TI host cell. In certain embodiments, the integration site is within an endogenous gene of the TI host cell.

In certain embodiments, the first exogenous nucleotide sequence is integrated at a safe harbor locus in the genome of the host cell. In certain embodiments, the first exogenous nucleotide sequence is integrated at a site within a specific locus of the genome of a TI host cell. In certain embodiments, the host cell is a human host cell, and the first exogenous nucleic acid sequence is integrated at an adeno-associated virus integration site.

In certain embodiments, the locus comprising the integration site of the first exogenous nucleotide sequence does not encode an open reading frame (ORF). In certain embodiments, the locus comprising the integration site of the first exogenous nucleotide sequence includes cis-acting elements, e.g., promoters and enhancers. In certain embodiments, the locus comprising the integration site of the exogenous nucleotide sequence is free of any cis-acting elements, e.g., promoters and enhancers, which enhance gene expression.

In certain embodiments, an integration site and/or the nucleotide sequences flanking the integration site can be identified experimentally. In certain embodiments, an integration site and/or the nucleotide sequences flanking the integration site can be identified by genome-wide screening approaches to isolate host cells that produce selectable markers and/or AAV at a desirable level. In certain embodiments, an integration site and/or the nucleotide sequences flanking an integration site can be identified by genome-wide screening approaches following transposase-based cassette integration event. In certain embodiments, an integration site and/or the nucleotide sequences flanking an integration site can be identified by brute force random integration screening. In certain embodiments, an integration site and/or the nucleotide sequences flanking an integration site can be determined by conventional sequencing approaches such as target locus amplification (TLA) followed by next-generation sequencing (NGS) and whole-genome NGS. In certain embodiments, the location of an integration site on a chromosome can be determined by conventional cell biology approaches such as fluorescence in-situ hybridization (FISH) analysis.

In certain embodiments, the integration sites can be on the same chromosome. In certain embodiments, the integration sites are located within 1-1,000 nucleotides, 1,000-100,000 nucleotides, 100,000-1,000,000 nucleotides or more from each other in the same chromosome. In certain embodiments the integration sites are on different chromosomes. In certain embodiments, a TI host cell comprising a first exogenous nucleotide sequence at one integration site can be used for the insertion of at least two, at least three, at least four, at least five, at least six, at least 7, at least 8, or more exogenous nucleotide sequences at the same or different integration sites.

In certain embodiments, targeted integration of the first exogenous nucleic acid sequence is by recombinase-mediated cassette exchange (RMCE). The feasibility of RMCE at multiple sites can be evaluated by methods known in the art, e.g., measuring the viral titer, capsid titer, or virus production. In certain embodiments, the evaluation can be performed by methods known in the art, e.g., by evaluating the titer and/or specific productivity of a culture of the TI host cell. Exemplary culture strategies include, but are not limited to, fed-batch shake flask cultures and a bioreactor fed-batch cultures. AAV titer and specific productivity of the TI host cells can be evaluated by methods known in the art, e.g., methods not limited to, droplet digital Polymerase Chain Reaction (ddPCR); Gyrolab® AAVX Titer, ELISA, FACS, Fluorometric Microvolume Assay Technology (FMAT), affinity chromatography, Western blot analysis.

### 4. Exogenous Nucleotide Sequences

An exogenous nucleotide sequence is a nucleotide sequence that does not originate from a host cell, but which can be introduced into a host cell by traditional DNA delivery methods, e.g., by transfection, electroporation, or transformation methods. In certain embodiments, the exogenous nucleotide sequence codes for proteins relevant to AAV production. In certain embodiments, the exogenous nucleotide sequences employed in the context of the instant disclosure comprises elements, e.g., nucleic acid sequences encoding one or more of adeno-associated virus (AAV) proteins REP, CAP, adenovirus (Ad) proteins E2A, Ad E4, E4orf6, E4orf6/7, E2DBP, or Ad VA, one or more recombination recognition sequences (RRSs) and a first selection marker. In certain embodiments, the first exogenous nucleotide sequences facilitating the introduction of additional nucleic acid sequences are referred to herein as “landing pads.” Accordingly, in certain embodiments, a TI host cell can comprise: (1) an exogenous nucleotide sequence that encodes for AAV or AAV-relevant gene products, e.g., proteins and/or RNA (e.g., Ad polypeptides and Ad VA RNA) incorporated into a particular locus in a host cell genome via targeted integration, e.g., an exogenous site-specific nuclease mediated (e.g., CRISPR/Cas9-mediated) targeted integration; (2) an exogenous nucleotide sequence that includes one or more landing pads; and/or (3) an exogenous nucleotide sequence that includes one or more landing pads into which one or more exogenous nucleotide sequences that encode for AAV or AAV-relevant polypeptides (e.g., Ad polypeptides) have been incorporated.

### 4.1 Landing Pads

In certain embodiments, a targeted integration first exogenous nucleotide sequence comprises one or more recombination recognition sequence (RRS), wherein the RRS can be recognized by a recombinase. In certain embodiments, the integrated first exogenous nucleotide sequence comprises at least two RRSs. In certain embodiments, the integrated first exogenous nucleotide sequence comprises two RRSs, which are the same. In certain embodiments, the integrated first exogenous nucleotide sequence comprises two RRSs, where the two RRSs are heterospecific, i.e., not recognized by the same recombinase. In certain embodiments, an integrated first exogenous nucleotide sequence comprises three RRSs, wherein the third RRS is located between the first and the second RRS. In certain embodiments, the first and the second RRS are the same and the third RRS is different from the first or the second RRS. In certain embodiments, all three RRSs are heterospecific. In certain embodiments, an integrated first exogenous nucleotide sequence comprises four, five, six, seven, or eight RRSs. In certain embodiments, an integrated first exogenous nucleotide sequence comprises multiple RRSs. In certain embodiments, the multiple two or more RRSs are the same. In certain embodiments, the two or more RRSs are heterospecific. In certain embodiments each RRS can be recognized by a distinct recombinase. In certain embodiments, the subset of the total number of RRSs are the homospecific, i.e., recognized by the same recombinase, and a subset of the total number of RRSs are heterospecific, i.e., not recognized by the same recombinase. In certain embodiments, the RRS or RRSs can be selected from the group consisting of a LoxP sequence, a LoxP L3 sequence, a LoxP 2L sequence, a LoxFas sequence, a Lox511 sequence, a Lox2272 sequence, a Lox2372 sequence, a Lox5171 sequence, a Loxm2 sequence, a Lox71 sequence, a Lox66 sequence, a flippase recognition target (FRT) sequence, a Bxb1 attP sequence, a Bxb1 attB sequence, a φC31 attP sequence, and a φC31 attB sequence.

In certain embodiments, the first exogenous nucleic acid sequence integrated in the genome of the TI host cells encodes one or more of an AAV replicase (REP) protein, an AAV capsid (CAP) protein, an adenovirus (Ad) E2A protein, Ad E4 protein, Ad E4orf6 protein, Ad E4orf6/7 protein, Ad E2DBP protein, or Ad VA RNA. In certain embodiments, the first exogenous nucleic acid sequence encodes AAV REP, Ad E2A, Ad E4, and Ad VA gene products, e.g., proteins and/or RNA. In certain embodiments, the first exogenous nucleic acid sequence encodes a first selection marker flanked by two recombination recognition sequences (RRSs). In certain embodiments, the first exogenous nucleic acid sequence encodes one or more of adeno-associated virus (AAV) REP, AAV CAP, adenovirus (Ad) E2A, Ad E4, Ad E4orf6, Ad E4orf6/7, Ad E2DBP, or Ad VA gene products, e.g., proteins and/or RNA, and a first selection marker flanked by two recombination recognition sequences (RRSs).

In certain embodiments, the integrated exogenous nucleotide sequence comprises three RRSs. In certain embodiments, the third RRS is located between the first and the second RRS. In certain embodiments, all three RRSs are the same. In certain embodiments, the first and the second RRS are the same, and the third RRS is different from the first or the second RRS. In certain embodiments, all three RRSs are heterospecific.

In certain embodiments, the integrated first exogenous nucleotide sequence comprises at least one selection marker. In certain embodiments, the integrated first exogenous nucleotide sequence comprises one RRS and at least one selection marker. In certain embodiments, the integrated first exogenous nucleotide sequence comprises a first and a second RRS, and at least one selection marker. In certain embodiments, a selection marker is located between the first and the second RRS. In certain embodiments, two RRSs flank at least one selection marker, i.e., a first RRS is located 5′ upstream and a second RRS is located 3′ downstream of the selection marker. In certain embodiments, a first RRS is adjacent to the 5′ end of the selection marker and a second RRS is adjacent to the 3′ end of the selection marker.

In certain embodiments, a selection marker is located between a first and a second RRS and the two flanking RRSs are the same. In certain embodiments, the two RRSs flanking the first selection marker are both LoxP sequences. In certain embodiments, the two RRSs flanking the first selection marker are both FRT sequences. In certain embodiments, a first selection marker is located between a first and a second RRS and the two flanking RRSs are heterospecific. In certain embodiments, the first flanking RRS is a LoxP L3 sequence and the second flanking RRS is a LoxP 2L sequence. In certain embodiments, a LoxP L3 sequenced is located 5′ of the first selection marker and a LoxP 2L sequence is located 3′ of the first selection marker. In certain embodiments, the first flanking RRS is a wild-type FRT sequence and the second flanking RRS is a mutant FRT sequence. In certain embodiments, the first flanking RRS is a Bxb1 attP sequence and the second flanking RRS is a Bxb1 attB sequence. In certain embodiments, the first flanking RRS is a QC31 attP sequence and the second flanking RRS is a φC31 attB sequence. In certain embodiments, the two RRSs are positioned in the same orientation. In certain embodiments, the two RRSs are both in the forward or reverse orientation. In certain embodiments, the two RRSs are positioned in opposite orientation.

In certain embodiments, a selection marker can be an aminoglycoside phosphotransferase (APH) (e.g., hygromycin phosphotransferase (HYG), neomycin and G418 APH), dihydrofolate reductase (DHFR), thymidine kinase (TK), glutamine synthetase (GS), asparagine synthetase, tryptophan synthetase (indole), histidinol dehydrogenase (histidinol D), and genes encoding resistance to puromycin, blasticidin, bleomycin, phleomycin, chloramphenicol, Zeocin, or mycophenolic acid. In certain embodiments, a selection marker can be a GFP, an eGFP, a synthetic GFP, a YFP, an eYFP, a CFP, an mPlum, an mCherry, a tdTomato, an mStrawberry, a J-red, a DsRed-monomer, an mOrange, an mKO, an mCitrine, a Venus, a YPet, an Emerald, a CyPet, an mCFPm, a Cerulean, or a T-Sapphire marker. In certain embodiments, the selection marker can be a fusion construct comprising at least two selection markers. In certain embodiments the gene encoding a selection marker, or a fragment of the selection marker can be fused to the gene encoding a different selection marker or a fragment thereof.

In certain embodiments, the integrated exogenous nucleotide sequence comprises two selection markers flanked by two RRSs, wherein a first selection marker is different from a second selection marker. In certain embodiments, the two selection markers are both selected from the group consisting of a glutamine synthetase selection marker, a thymidine kinase selection marker, a HYG selection marker, and a puromycin resistance selection marker. In certain embodiments, the integrated exogenous nucleotide sequence comprises a thymidine kinase selection marker and a HYG selection marker. In certain embodiments, the first selection maker is selected from the group consisting of an aminoglycoside phosphotransferase (APH) (e.g., hygromycin phosphotransferase (HYG), neomycin and G418 APH), dihydrofolate reductase (DHFR), thymidine kinase (TK), glutamine synthetase (GS), asparagine synthetase, tryptophan synthetase (indole), histidinol dehydrogenase (histidinol D), and genes encoding resistance to puromycin, blasticidin, bleomycin, phleomycin, chloramphenicol, Zeocin, and mycophenolic acid, and the second selection maker is selected from the group consisting of a GFP, an eGFP, a synthetic GFP, a YFP, an eYFP, a CFP, an mPlum, an mCherry, a tdTomato, an mStrawberry, a J-red, a DsRed-monomer, an mOrange, an mKO, an mCitrine, a Venus, a YPet, an Emerald, a CyPet, an mCFPm, a Cerulean, and a T-Sapphire marker. In certain embodiments, the first selection marker is a glutamine synthetase selection marker, and the second selection marker is a GFP marker. In certain embodiments, the two RRSs flanking both selection markers are the same. In certain embodiments, the two RRSs flanking both selection markers are different.

In certain embodiments, the selection marker is operably linked to a promoter sequence. In certain embodiments, the selection marker is operably linked to an SV40 promoter. In certain embodiments, the selection marker is operably linked to a Cytomegalovirus (CMV) promoter.

In certain embodiments, the integrated exogenous nucleotide sequence comprises at least one selection marker and an IRES, wherein the IRES is operably linked to the selection marker. In certain embodiments, the selection marker operably linked to the IRES is selected from the group consisting of a GFP, an eGFP, a synthetic GFP, a YFP, an eYFP, a CFP, an mPlum, an mCherry, a tdTomato, an mStrawberry, a J-red, a DsRed-monomer, an mOrange, an mKO, an mCitrine, a Venus, a YPet, an Emerald, a CyPet, an mCFPm, a Cerulean, and a T-Sapphire marker. In certain embodiments, the selection marker operably linked to the IRES is a GFP marker. In certain embodiments, the integrated exogenous nucleotide sequence comprises an IRES, and two selection markers flanked by two RRSs, wherein the IRES is operably linked to the second selection marker. In certain embodiments, the integrated exogenous nucleotide sequence comprises an IRES, and three selection markers flanked by two RRSs, wherein the IRES is operably linked to the third selection marker. In certain embodiments, the integrated exogenous nucleotide sequence comprises an IRES, and three selection markers flanked by two RRSs, wherein the IRES is operably linked to the third selection marker. In certain embodiments, the third selection marker is different from the first or the second selection marker. In certain embodiments, the integrated exogenous nucleotide sequence comprises a first selection marker operably linked to a promoter and a second selection marker operably linked to an IRES. In certain embodiments, the integrated exogenous nucleotide sequence comprises a glutamine synthetase selection marker operably linked to a SV40 promoter and a GFP selection marker operably linked to an IRES. In certain embodiments, the integrated exogenous nucleotide sequence comprises a thymidine kinase selection marker and a HYG selection marker operably linked to a CMV promoter and a GFP selection marker operably linked to an IRES.

In certain embodiments, the stable mammalian host cells comprise a second exogenous nucleic acid sequence integrated at least once in the genome of the stable mammalian cell. In some embodiments, integration of the second exogenous nucleic acid sequence is mediated by a transposon.

In certain embodiments, the second exogenous nucleic acid sequence comprises ITRs flanking a gene of interest (GOI) coding sequences for a polypeptide of interest (POI), and a second selection marker. In certain embodiments, the one of more GOI comprise coding sequences for one or more of AAV REP, AAV CAP, Ad E2A, Ad E4, Ad E4orf6, Ad E4orf6/7, Ad E2DBP, or Ad VA gene products, e.g., proteins and/or RNA. In certain embodiments, the mammalian cell comprises one to ten additional exogenous nucleic acids comprising ITRs flanking coding sequences for one or more of AAV REP, AAV CAP, Ad E2A, Ad E4, Ad E4orf6, Ad E4orf6/7, Ad E2DBP, or Ad VA, and a selection marker, where the one to ten additional exogenous nucleic acids are each integrated at least once in the genome of the mammalian cell. In certain embodiments, the one to ten additional exogenous nucleic acids each comprise distinct selection markers. In certain embodiments, two or more of the one to ten additional exogenous nucleic acids comprise the same selection marker. In certain embodiments, one or more of the additional exogenous nucleic acids can further comprise a coding sequence for a POI within the flanking ITRs.

In certain embodiments, the one or more of the one to ten additional exogenous nucleic acids further comprise a coding sequence for a gene of interest (GOI) within the flanking inverted terminal repeats.

In certain embodiments, the stable mammalian host cell comprises one to ten additional exogenous nucleic acids each integrated at least once in the genome of the stable mammalian cell. In certain embodiments, the one to ten additional exogenous nucleic acids comprise inverted terminal repeats flanking coding sequences for one or more of AAV REP, AAV CAP, Ad E2A, Ad E4, Ad E4orf6, Ad E4orf6/7, Ad E2DBP, or Ad VA and a selection marker. In certain embodiments, the one to ten additional exogenous nucleic acids each comprise distinct selection markers. In certain embodiments, two or more of the one to ten additional exogenous nucleic acids comprise the same selection marker. In certain embodiments, the one or more of the one to ten additional exogenous nucleic acids further comprise a coding sequence for a polypeptide of interest (POI) within the flanking inverted terminal repeats.

In certain embodiments, the stable mammalian host cell further comprises a coding sequence for a second polypeptide of interest within the flanking inverted terminal repeats.

In certain embodiments, the stable mammalian host cell further comprises an expression cassette of transactivator (rTA) sequence, wherein the rTA sequence is fused to the first and/or the second selection marker. In certain embodiments, the rTA is fused to the first and/or the second selection marker via a 2A peptide. For example, but not by way of limitation, the 2A peptide can be selected from P2A, T2A, E2A, and F2A. In certain embodiments, the rTA is fused to the first and/or the second selection marker via a P2A peptide sequence.

In certain embodiments, a selection marker can be an aminoglycoside phosphotransferase (APH) (e.g., hygromycin phosphotransferase (HYG), neomycin and G418 APH), dihydrofolate reductase (DHFR), thymidine kinase (TK), glutamine synthetase (GS), asparagine synthetase, tryptophan synthetase (indole), histidinol dehydrogenase (histidinol D), and genes encoding resistance to puromycin, blasticidin, bleomycin, phleomycin, chloramphenicol, Zeocin, or mycophenolic acid. In certain embodiments, a selection marker can be a GFP, an eGFP, a synthetic GFP, a YFP, an eYFP, a CFP, an mPlum, an mCherry, a tdTomato, an mStrawberry, a J-red, a DsRed-monomer, an mOrange, an mKO, an mCitrine, a Venus, a YPet, an Emerald, a CyPet, an mCFPm, a Cerulean, or a T-Sapphire marker. In certain embodiments, the selection marker can be a fusion construct comprising at least two selection markers. In certain embodiments the gene encoding a selection marker, or a fragment of the selection marker can be fused to the gene encoding a different selection marker or a fragment thereof.

In certain embodiments, the integrated exogenous nucleotide sequence comprises two selection markers flanked by two ITRs, wherein a first selection marker is different from a second selection marker. In certain embodiments, the two selection markers are both selected from the group consisting of a glutamine synthetase selection marker, a thymidine kinase selection marker, a HYG selection marker, and a puromycin resistance selection marker. In certain embodiments, the integrated exogenous nucleotide sequence comprises a thymidine kinase selection marker and a HYG selection marker. In certain embodiments, the first selection maker is selected from the group consisting of an aminoglycoside phosphotransferase (APH) (e.g., hygromycin phosphotransferase (HYG), neomycin and G418 APH), dihydrofolate reductase (DHFR), thymidine kinase (TK), glutamine synthetase (GS), asparagine synthetase, tryptophan synthetase (indole), histidinol dehydrogenase (histidinol D), and genes encoding resistance to puromycin, blasticidin, bleomycin, phleomycin, chloramphenicol, Zeocin, and mycophenolic acid, and the second selection maker is selected from the group consisting of a GFP, an eGFP, a synthetic GFP, a YFP, an eYFP, a CFP, an mPlum, an mCherry, a tdTomato, an mStrawberry, a J-red, a DsRed-monomer, an mOrange, an mKO, an mCitrine, a Venus, a Ypet, an Emerald, a CyPet, an mCFPm, a Cerulean, and a T-Sapphire marker. In certain embodiments, the first selection marker is a glutamine synthetase selection marker, and the second selection marker is a GFP marker. In certain embodiments, the two ITRs flanking both selection markers are the same. In certain embodiments, the two ITRs flanking both selection markers are different.

In certain embodiments, the selection marker is operably linked to a promoter sequence. In certain embodiments, the selection marker is operably linked to an SV40 promoter. In certain embodiments, the selection marker is operably linked to a Cytomegalovirus (CMV) promoter.

In certain embodiments, the integrated exogenous nucleotide sequence comprises at least one selection marker and an IRES, wherein the IRES is operably linked to the selection marker. In certain embodiments, the selection marker operably linked to the IRES is selected from the group consisting of a GFP, an eGFP, a synthetic GFP, a YFP, an eYFP, a CFP, an mPlum, an mCherry, a tdTomato, an mStrawberry, a J-red, a DsRed-monomer, an mOrange, an mKO, an mCitrine, a Venus, a Ypet, an Emerald, a CyPet, an mCFPm, a Cerulean, and a T-Sapphire marker. In certain embodiments, the selection marker operably linked to the IRES is a GFP marker. In certain embodiments, the integrated exogenous nucleotide sequence comprises an IRES, and two selection markers flanked by two ITRs, wherein the IRES is operably linked to the second selection marker. In certain embodiments, the integrated exogenous nucleotide sequence comprises an IRES, and three selection markers flanked by two ITRs, wherein the IRES is operably linked to the third selection marker. In certain embodiments, the integrated exogenous nucleotide sequence comprises an IRES, and three selection markers flanked by two ITRs, wherein the IRES is operably linked to the third selection marker. In certain embodiments, the third selection marker is different from the first or the second selection marker. In certain embodiments, the integrated exogenous nucleotide sequence comprises a first selection marker operably linked to a promoter and a second selection marker operably linked to an IRES. In certain embodiments, the integrated exogenous nucleotide sequence comprises a glutamine synthetase selection marker operably linked to a SV40 promoter and a GFP selection marker operably linked to an IRES. In certain embodiments, the integrated exogenous nucleotide sequence comprises a thymidine kinase selection marker and a HYG selection marker operably linked to a CMV promoter and a GFP selection marker operably linked to an IRES.

### 5. Methods of Targeted Integration and Transposon-Mediated Genomic Integration

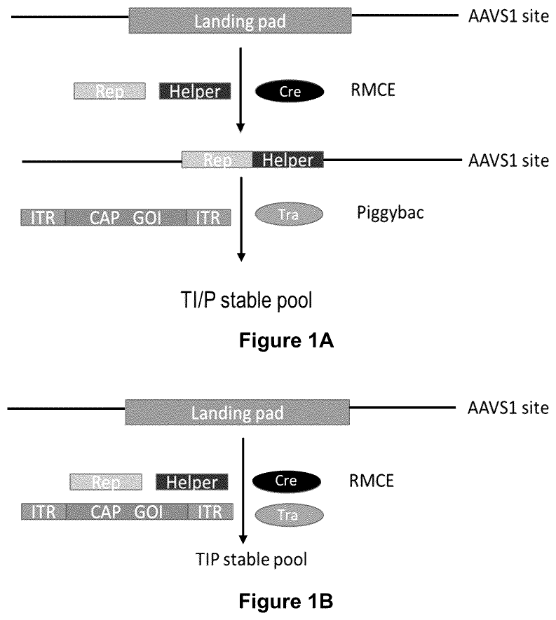

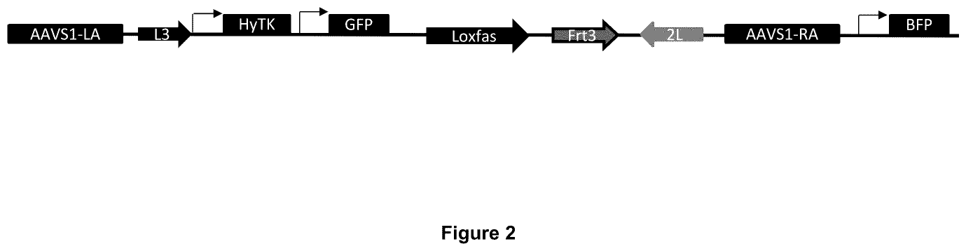

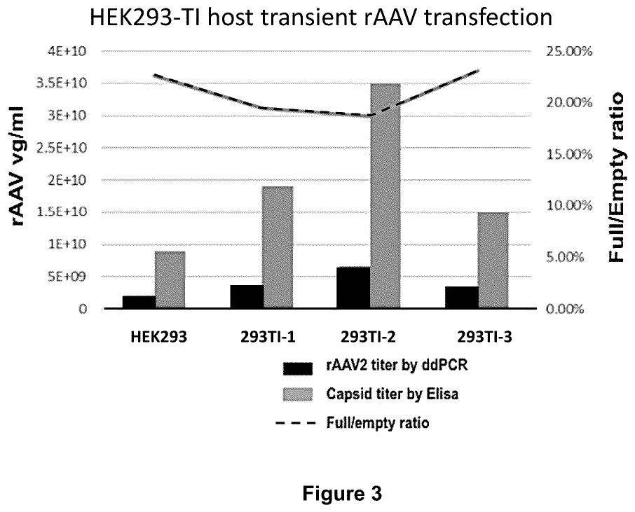

In certain embodiments, targeted integration (TI) can be combined with transposon-mediated genomic integration. In certain embodiments, the targeted integration can be followed by transposon-mediated genomic integration. In certain embodiments, the targeted integration can be performed concurrently with transposon-mediated genomic integration. In certain embodiments, the targeted integration can be followed by transposon-mediated genomic integration. FIGS. 1A-1B depict exemplary TIP and TI/P strategies for targeted integration and transposon-mediated integration. FIG. 2 shows an exemplary construct for targeted integration and transposon-mediated integration of one or more AAV production genes into the genome of a mammalian host cell.

### 5.1 Targeted Integration via Recombinase-Mediated Recombination

A “recombination recognition sequence” (RRS) is a nucleotide sequence recognized by a recombinase and is necessary and sufficient for recombinase-mediated recombination events. A RRS can be used to define the position where a recombination event will occur in a nucleotide sequence.

In certain embodiments, a RRS is selected from the group consisting of a LoxP sequence, a LoxP L3 sequence, a LoxP 2L sequence, a LoxFas sequence, a Lox511 sequence, a Lox2272 sequence, a Lox2372 sequence, a Lox5171 sequence, a Loxm2 sequence, a Lox71 sequence, a Lox66 sequence, a FRT sequence, a Bxb1 attP sequence, a Bxb1 attB sequence, a φC31 attP sequence, and a φC31 attB sequence.

In certain embodiments, a RRS can be recognized by a Cre recombinase. In certain embodiments, a RRS can be recognized by a FLP recombinase. In certain embodiments, a RRS can be recognized by a Bxb1 integrase. In certain embodiments, a RRS can be recognized by a PC31 integrase.

In certain embodiments when the RRS is a LoxP site, the host cell requires the Cre recombinase to perform the recombination. In certain embodiments when the RRS is a flippase recognition target (FRT) site, the host cell requires the FLP recombinase to perform the recombination. In certain embodiments when the RRS is a Bxb1 attP or a Bxb1 attB site, the host cell requires the Bxb1 integrase to perform the recombination. In certain embodiments when the RRS is a φC31 attP or a φC31attB site, the host cell requires the φC31 integrase to perform the recombination. The recombinases can be introduced into a host cell using an expression vector comprising coding sequences of the enzymes.

The Cre-LoxP site-specific recombination system has been widely used in many biological experimental systems. Cre is a 38-kDa site-specific DNA recombinase that recognizes 34 bp LoxP sequences. Cre is derived from bacteriophage P1 and belongs to the tyrosine family site-specific recombinase. Cre recombinase can mediate both intra and intermolecular recombination between LoxP sequences. The LoxP sequence is composed of an 8 bp nonpalindromic core region flanked by two 13 bp inverted terminal repeats. Cre recombinase binds to the 13 bp repeat thereby mediating recombination within the 8 bp core region. Cre-LoxP-mediated recombination occurs at a high efficiency and does not require any other host factors. If two LoxP sequences are placed in the same orientation on the same nucleotide sequence, Cre-mediated recombination will excise DNA sequences located between the two LoxP sequences as a covalently closed circle. If two LoxP sequences are placed in an inverted position on the same nucleotide sequence, Cre-mediated recombination will invert the orientation of the DNA sequences located between the two sequences. LoxP sequences can also be placed on different chromosomes to facilitate recombination between different chromosomes. If two LoxP sequences are on two different DNA molecules and if one DNA molecule is circular, Cre-mediated recombination will result in integration of the circular DNA sequence.

In certain embodiments, a LoxP sequence is a wild-type LoxP sequence. In certain embodiments, a LoxP sequence is a mutant LoxP sequence. Mutant LoxP sequences have been developed to increase the efficiency of Cre-mediated integration or replacement. In certain embodiments, a mutant LoxP sequence is selected from the group consisting of a LoxP L3 sequence, a LoxP 2L sequence, a LoxFas sequence, a Lox511 sequence, a Lox2272 sequence, a Lox2372 sequence, a Lox5171 sequence, a Loxm2 sequence, a Lox71 sequence, and a Lox66 sequence. For example, the Lox71 sequence has 5 bp mutated in the left 13 bp repeat. The Lox66 sequence has 5 bp mutated in the right 13 bp repeat. Both the wild-type and the mutant LoxP sequences can mediate Cre-dependent recombination.

The FLP-FRT site-specific recombination system is similar to the Cre-Lox system. It involves the flippase (FLP) recombinase, which is derived from the 2 μm plasmid of the yeast Saccharomyces cerevisiae. FLP also belongs to the tyrosine family site-specific recombinase. The FRT sequence is a 34 bp sequence that consists of two palindromic sequences of 13 bp each flanking an 8 bp spacer. FLP binds to the 13 bp palindromic sequences and mediates DNA break, exchange and ligation within the 8 bp spacer. Similar to the Cre recombinase, the position and orientation of the two FRT sequences determine the outcome of FLP-mediated recombination. In certain embodiments, a FRT sequence is a wild-type FRT sequence. In certain embodiments, a FRT sequence is a mutant FRT sequence. Both the wild-type and the mutant FRT sequences can mediate FLP-dependent recombination. In certain embodiments, a FRT sequence is fused to a responsive receptor domain sequence, such as, but not limited to, a tamoxifen responsive receptor domain sequence.

Bxb1 and φC31 belong to the serine recombinase family. They are both derived from bacteriophages and are used by these bacteriophages to establish lysogeny to facilitate site-specific integration of the phage genome into the bacterial genome. These integrases catalyze site-specific recombination events between short (40-60 bp) DNA substrates termed attP and attB sequences that are originally attachment sites located on the phage DNA and bacterial DNA, respectively. After recombination, two new sequences are formed, which are termed attL and attR sequences and each contains half sequences derived from attP and attB. Recombination can also occur between attL and attR sequences to excise the integrated phage out of the bacterial DNA. Both integrases can catalyze the recombination without the aid of any additional host factors. In the absence of any accessory factors, these integrases mediate unidirectional recombination between attP and attB with greater than 80% efficiency. Because of the short DNA sequences that can be recognized by these integrases and the unidirectional recombination, these recombination systems have been developed as a complement to the widely used Cre-LoxP and FRT-FLP systems for genetic engineering purposes.

The terms “matching RRSs” and “homospecific RRSs” indicates that a recombination occurs between two RRSs. In certain embodiments, the two matching RRSs are the same. In certain embodiments, both RRSs are wild-type LoxP sequences. In certain embodiments, both RRSs are mutant LoxP sequences. In certain embodiments, both RRSs are wild-type FRT sequences. In certain embodiments, both RRSs are mutant FRT sequences. In certain embodiments, the two matching RRSs are different sequences but can be recognized by the same recombinase. In certain embodiments, the first matching RRS is a Bxb1 attP sequence and the second matching RRS is a Bxb1 attB sequence. In certain embodiments, the first matching RRS is a φC31 attB sequence and the second matching RRS is a φC31 attB sequence.

In certain embodiments, an integrated exogenous nucleotide sequence comprises two RRSs and a vector comprises two RRSs matching the two RRSs on the integrated exogenous nucleotide sequence, i.e., the first RRS on the integrated exogenous nucleotide sequence matches the first RRS on the vector and the second RRS on the integrated exogenous nucleotide sequence matches the second RRS on the vector. In certain embodiments, the first RRS on the integrated exogenous nucleotide sequence and the first RRS on the vector are the same as the second RRS on the integrated exogenous nucleotide sequence and the second RRS on the vector. A non-limiting example of such a “single-vector RMCE” strategy is presented in FIG. 2A of PCT Application PCT/US2018/067070 (Publication No. WO2019126634). In certain embodiments, the first RRS on the integrated exogenous nucleotide sequence and the first RRS on the vector are different from the second RRS on the integrated exogenous nucleotide sequence and the second RRS on the vector. In certain embodiments, the first RRS on the integrated exogenous nucleotide sequence and the first RRS on the vector are both LoxP L3 sequences, and the second RRS on the integrated exogenous nucleotide sequence and the second RRS on the vector are both LoxP 2L sequences.

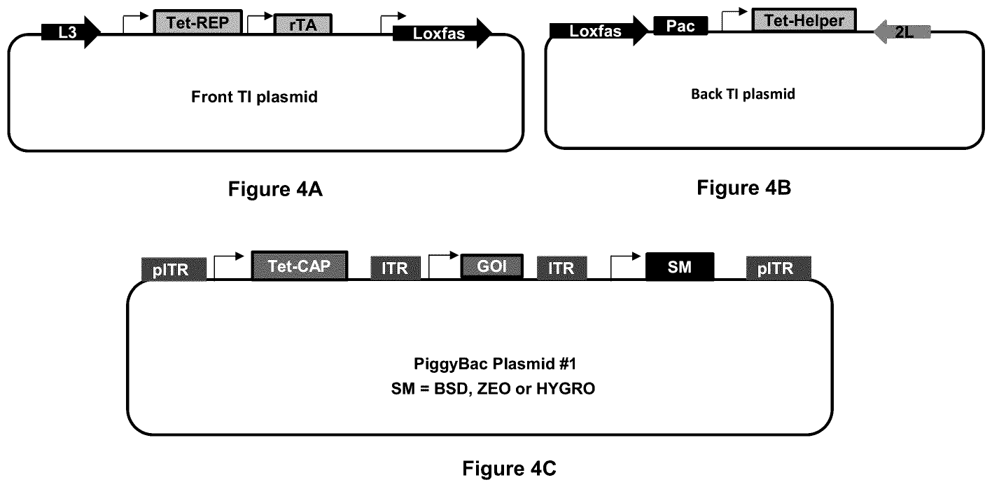

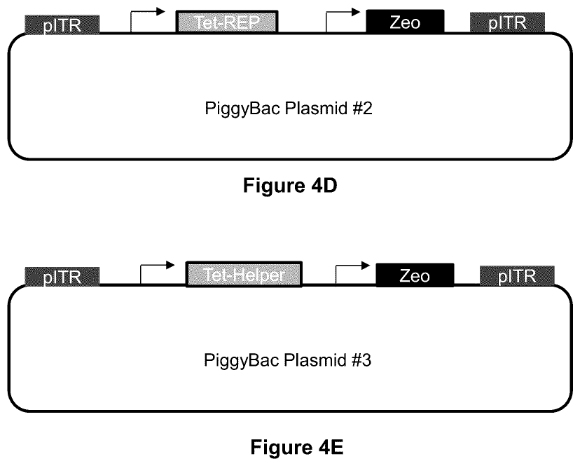

In certain embodiments, a “two-vector RMCE” strategy is employed. For example, but not by way of limitation, an integrated exogenous nucleotide sequence could comprise three RRSs, e.g., an arrangement where the third RRS (“RRS3”) is present between the first RRS (“RRS1”) and the second RRS (“RRS2”), while a first vector comprises two RRSs matching the first and the third RRS on the integrated exogenous nucleotide sequence, and a second vector comprises two RRSs matching the third and the second RRS on the integrated exogenous nucleotide sequence. An example of a two vector RMCE strategy is illustrated in FIG. 4 of PCT Application PCT/US2018/067070 (Publication No. WO2019126634). In such an example, RRS1, RRS2, and RRS3 are heterospecific, e.g., they do not cross-react with each other. In some embodiments, one vector (front) comprises the RRS1, and a promoter followed by a start codon and RRS3 (in this order). The other vector (back) comprises the RRS3 fused to the coding sequence of a marker without the start codon (ATG), a GOI and the RRS2 (in this order). Additional nucleotides may be inserted between the RRS3 site and the selection marker sequence to ensure in frame translation for the fusion protein.

Both single-vector and two-vector RMCE allow for unidirectional integration of one or more donor DNA molecule(s) into a pre-determined site of a host cell genome, and precise exchange of a DNA cassette present on the donor DNA with a DNA cassette on the host genome where the integration site resides. The DNA cassettes are characterized by two heterospecific RRSs flanking at least one selection marker (although in certain two-vector RMCE examples a “split selection marker” can be used as outlined herein) and/or at least one exogenous GOI. RMCE involves double recombination cross-over events, catalyzed by a recombinase, between the two heterospecific RRSs within the target genomic locus and the donor DNA molecule. RMCE is designed to introduce AAV genes, Ad helper genes, and/or selection markers into the pre-determined locus of a host cell genome. Unlike recombination which involves just one cross-over event, RMCE can be implemented such that prokaryotic vector sequences are not introduced into the host cell genome, thus reducing and/or preventing unwanted triggering of host immune or defense mechanisms. The RMCE procedure can be repeated with multiple DNA cassettes.

In certain embodiments, targeted integration is achieved by one cross-over recombination event, wherein one exogenous nucleotide sequence comprising one RRS adjacent to at least one exogenous gene encoding for AAV proteins, Ad helper proteins, and/or at least one selection marker is integrated into a pre-determined site of a host cell genome. In certain embodiments, targeted integration is achieved by one RMCE, wherein a DNA cassette comprising at least an exogenous AAV gene, Ad helper gene, and/or at least one selection marker flanked by two heterospecific RRSs is integrated into a pre-determined site of a host cell genome. In certain embodiments, targeted integration is achieved by two RMCEs, wherein two different DNA cassettes, each comprising at least an exogenous AAV gene, Ad helper gene, and/or at least one selection marker flanked by two heterospecific RRSs, are both integrated into a pre-determined site of a host cell genome. In certain embodiments, targeted integration is achieved by multiple RMCEs, wherein DNA cassettes from multiple vectors, each comprising at least an exogenous AAV gene, Ad helper gene, and/or at least one selection marker flanked by two heterospecific RRSs, are all integrated into a pre-determined site of a host cell genome. In certain embodiments the selection marker can be partially encoded on the first the vector and partially encoded on the second vector such that the integration of both RMCEs allows for the expression of the selection marker. An example of such a system is presented in FIG. 4 of PCT Application PCT/US2018/067070 (Publication No. WO2019126634).

In certain embodiments, targeted integration via recombinase-mediated recombination leads to a selection marker or one or more exogenous AAV gene, Ad helper gene, and/or at least one selection marker integrated into one or more pre-determined integration sites of a host cell genome along with sequences from a prokaryotic vector. In certain embodiments, targeted integration via recombinase-mediated recombination leads to AAV genes, Ad helper genes, and/or a selection marker being integrated into one or more pre-determined integration sites of a host cell genome free of sequences from a prokaryotic vector.

### 5.2 Targeted Integration Via Homologous Recombination, HDR, or NHEJ

The presently disclosed subject matter also relates to targeted integration mediated by homologous recombination or by an exogenous site-specific nuclease followed by HDR or NHEJ.

Homologous recombination is a recombination between DNA molecules that share extensive sequence homology. It can be used to direct error-free repair of double-stranded DNA breaks and generates sequence variation in gametes during meiosis. Since homologous recombination involves the exchange of genetic information between two homologous DNA molecules, it does not alter the overall arrangement of the genes on a chromosome. During homologous recombination, a nick or break forms in double-stranded DNA (dsDNA), followed by the invasion of a homologous dsDNA molecule by a single-stranded DNA end, pairing of homologous sequences, branch migration to form a Holliday junction, and final resolution of the Holliday junction.

Double-strand break (DSB) is the most severe form of DNA damage and repair of such DNA damage is essential for the maintenance of genome integrity in all organisms. There are two major repair pathways to repair DSBs. The first repair pathway is homology-directed repair (HDR) pathway and homologous recombination is the most common form of HDR. Since HDR requires the presence of homologous DNA present in the cell, this repair pathway is normally active in S and G2 phase of the cell cycle wherein newly replicated sister chromatids are available as homologous templates. HDR is also a major repair pathway to repair collapsed replication forks during DNA replication. HDR is considered as a relatively error-free repair pathway. The second repair pathway for DSBs is non-homologous end joining (NHEJ). NHEJ is a repair pathway wherein the ends of a broken DNA are ligated together without the requirement for a homologous DNA template.

Targeted integration can be facilitated by exogenous site-specific nucleases followed by HDR. This is due to that the frequency of homologous recombination can be increased by introducing a DSB at a specific target genomic site. In certain embodiments, an exogenous nuclease can be selected from the group consisting of a zinc finger nuclease (ZFN), a ZFN dimer, a transcription activator-like effector nuclease (TALEN), a TAL effector domain fusion protein, an RNA-guided DNA endonuclease, an engineered meganuclease, and a clustered regularly interspaced short palindromic repeats (CRISPR)-associated (Cas) endonuclease.

CRISPR/Cas and TALEN systems are two genome editing tools that offer the best ease of construction and high efficiency. CRISPR/Cas was identified as an immune defense mechanism of bacteria against invading bacteriophages. Cas is a nuclease that, when guided by a synthetic guide RNA (gRNA), is capable of associating with a specific nucleotide sequence in a cell and editing the DNA in or around that nucleotide sequence, for instance by making one or more of a single-strand break, a DSB, and/or a point mutation. TALEN is an engineered site-specific nuclease, which is composed of the DNA-binding domain of TALE (transcription activator-like effectors) and the catalytic domain of restriction endonuclease FokI. By changing the amino acids present in the highly variable residue region of the monomers of the DNA binding domain, different artificial TALENs can be created to target various nucleotides sequences. The DNA binding domain subsequently directs the nuclease to the target sequences and creates a DSB.

Targeted integration via homologous recombination or HDR involves the presence of homologous sequences to the integration site. In certain embodiments, the homologous sequences are present on a vector. In certain embodiments, the homologous sequences are present on a polynucleotide.

In certain embodiments, homologous recombination is carried out without any accessory factors. In certain embodiments, homologous recombination is facilitated by the presence of vectors that are capable of integration. For example, in the context of the instant disclosure the integrating vector is an adeno-associated virus vector.

### 5.3 Transposon-Mediated Genomic Integration

The presently disclosed subject matter relates to TI host cells that are also subjected to transposon-mediated genomic integration of one or more exogenous nucleic acids, as well as methods of producing and using said combined transposon-mediated genomic integration and TI host cells. As outlined herein, in certain embodiments, the targeted integration can be performed concurrently with transposon-mediated genomic integration. In certain embodiments, the targeted integration can be followed by transposon-mediated genomic integration. In certain embodiments the targeted integration can be preceded by transposon-mediated genomic integration.

Transposons are useful in connection with the methods described herein. Exemplary transposons that may be used include, but are not limited to piggyBac, Sleeping Beauty, Tol2 and variants thereof;
(i) The natural insect-derived piggyBac transposable element (Wilson et. Al., Molecular Therapy, 15 (1): 139-145 (2007)) allows for “cut and paste” transposition of a transgene into the genome at target integration sites (e.g., TTAA) enabled by a transposase. Studies using human cells have revealed that piggyBac mediated transposition is efficient and has several advantages including, non-random integration site selectivity, precise excision (i.e., absence of “footprint” mutation in the donor plasmid and genomic DNA), high frequency of integrations, and absence of overproduction inhibition. These benefits coupled with its ability to deliver large transposable elements from ˜9.1 kb to ˜14.3 kb without a significant reduction in efficiency makes it one of the transposons of choice for gene therapy.
(ii) The engineered Tc1/mariner transposon Sleeping Beauty (Ivics et al., Curr. Issues Mol. Biol. 2004, 6 (1), 43-56; Ivics et al. Cell 91:501-510 (1997)) enables transposition of a gene cargo (2-kb to 10-kb) at target integration sites (e.g., TA dinucleotide; random). Unlike the piggyBac transposon, gene transposition by the Sleeping Beauty transposon is characterized by loss in transposition efficiency with larger transgenes, predictable “footprint” mutation in the donor plasmid and genomic DNA and sensitivity to overproduction inhibition.
(iii) The natural medaka fish hAT gene family transposable element Tol2 (Balciunas et al., PloS Genent. November 10; 2(11):e169 (2006)), and its variant, miniTol2 show high potential for use in mammalian gene transpositions. Similar to piggyBac, Tol2 offers many advantages including, absence of overproduction inhibition and large cargo-capacity (2-kb to 10-kb). Similar to Sleeping Beauty, Tol2 shows random integration capability.

In general, the transposons useful in connection with the methods of the instant disclosure translocate via a non-replicative, ‘cut-and-paste’ mechanism. For example, while not being bound by theory, transposition catalyzed by transposons useful in connection with the methods described herein, can proceed by the recognition of two terminal inverted terminal repeats (ITRs) by a DNA transposase that cleaves its target and consequently releases the DNA transposon from its donor sequence (e.g., donor plasmid). Upon excision, the transposons can integrate into the host cell genome cleaved by the same transposase at a corresponding sequence within the genome.

### 5.4 Regulated Expression of Proteins Involved in AAV Production

There are many cases where protein expression levels are not optimal mainly because the encoded proteins are difficult-to-express. The low expression level of difficult-to-express proteins can have diverse and difficult to identify causes. One possibility is the toxicity of the expressed proteins in the host cells. In such cases, a regulated expression system can be used to express toxic proteins where the sequences of interest encoding the proteins are under the control of an inducible promoter. In these systems, expression of the difficult-to-express proteins is only prompted when a regulator, e.g., small molecule, such as, but not limited to, tetracycline or its analogue, doxycycline (DOX), is added to the culture. Regulating the expression of toxic proteins (e.g., Rep) could alleviate the toxic effects, allowing the cultures to achieve the desired cell growth prior to production. In certain embodiments, a regulated expression system comprises at least one protein that is transcribed under a regulated promoter operably linked thereto. In certain embodiments, regulated expression system can be used to determine the underlying causes of low AAV production, or low protein expression for a difficult-to-express polypeptide of interest (POI). In certain embodiments, the ability to selectively turn off the expression of a protein in a regulated expression system can be used to link expression of a protein to an observed adverse effect.

In certain embodiments, to minimize transcriptional and cell line variability effects during the root cause analysis of difficult-to-express molecules, a regulated expression system can be used. For example, but not by way of limitation, the expression of the protein in a combined transposon-mediated genomically integrated and TI host can be triggered by addition to the culture of a regulator (inducer), e.g., doxycycline. In certain embodiments, the regulated expression vector utilizes a tetracycline-regulated promoter to express the protein, allowing for regulated expression of the protein.

In certain embodiments, the regulated expression system described in the present disclosure can be used to successfully determine the underlying cause(s) of low protein expression as compared to control cell line. In certain embodiments, once the lower relative expression of a protein in a regulated expression cell line is confirmed, the intracellular accumulation and secretion levels of the protein can be evaluated by leveraging protein translation inhibitor treatments, e.g., Dox and cycloheximide.

As outlined in detail herein, regulated expression can be based on gene switches for blocking or activating mRNA synthesis by regulated coupling of transcriptional repressors or activators to constitutive or minimal promoters. In certain non-limiting embodiments, repression can be achieved by binding the repressor proteins, e.g., where the proteins sterically block transcriptional initiation, or by actively repressing transcription through transcriptional silencers. In certain non-limiting embodiments, activation of mammalian or viral enhancerless minimal promoters can be achieved by the regulated coupling to an activation domain.

In certain embodiments, the conditional coupling of transcriptional repressors or activators can be achieved by using allosteric proteins that bind the promoters in response to external stimuli. In certain embodiments, the conditional coupling of transcriptional repressors or activators can be achieved by using intracellular receptors that are released from sequestrating proteins and, thus, can bind target promoters. In certain embodiments, the conditional coupling of transcriptional repressors or activators can be achieved by using chemically induced dimerizers.

In certain embodiments, the allosteric proteins used in the regulated expression systems of the present disclosure can be proteins that modulate transcriptional activity in response to antibiotics, bacterial quorum-sensing messengers, catabolites, or to the cultivation parameters, such as temperature, e.g., cold or heat. In certain embodiments, such regulated expression systems can be catabolite-based, e.g., where a bacterial repressor that controls catabolic genes for alternative carbon sources has been transferred to mammalian cells. In certain embodiments, the repression of the target promoter can be achieved by cumate-responsive binding of the repressor CymR. In certain embodiments, the catabolite-based system can rely on the activation of chimeric promoters by 6-hydroxynicotine-responsive binding of the prokaryotic repressor HdnoR, fused to the Herpes simplex VP16 transactivation domain.

In certain embodiments, the regulated expression system of the present disclosure can employ a quorum-sensing-based expression system originated from prokaryotes that manage intra- and inter-population communication by quorum-sensing molecules. These quorum-sensing molecules bind to receptors in target cells, modulate the receptors' affinity to cognate promoters leading to the initiation of specific regulon switches. In certain embodiments, the quorum-sensing molecule can be the N-(3-oxo-octanoyl)-homoserine lactone in the presence of which, the TraR-p65 fusion protein activates expression from a minimal promoter fused to the TraR-specific operator sequence. In certain embodiments, the quorum-sensing molecule can be the butyrolactone SCB1 (racemic 2-(1′-hydroxy-6-methylheptyl)-3-(hydroxymethyl)-butanolide) in a system based on the Streptomyces coelicolor A3 (2) ScbR repressor that binds its cognate operator OscbR in the absence of the SCB1. In certain embodiments, the quorum-sensing molecule can be homoserine-derived inducers used in a RTI system wherein Pseudomonas aeruginosa quorum-sensing repressors RhlR and LasR are fused to the SV40 T-antigen nuclear localization sequence and the Herpes simplex VP16 domain and can activate promoters containing specific operator sequences (las boxes).

In certain embodiments, the inducing molecules that modulate the allosteric proteins used in the regulated expression systems of the present disclosure can be, but are not limited to, cumate, isopropyl-β-D-thiogalactopyranoside (IPTG), macrolides, 6-hydroxynicotine, doxycycline, streptogramins, NADH, tetracycline.

In certain embodiments, the intracellular receptors used in the regulated expression systems of the present disclosure can be cytoplasmic or nuclear receptors. In certain embodiments, the regulated expression systems of the present disclosure can utilize the release of transcription factors from sequestering and inhibiting proteins by using small molecules. In certain embodiments, the regulated expression systems of the present disclosure can rely on steroid-regulation, wherein a hormone receptor is fused to a natural or an artificial transcription factor that can be released from HSP90 in the cytosol, migrate into the nucleus and activate selected promoters. In certain embodiments, mutant receptors can be used that are regulated by synthetic steroid analogs in order to avoid crosstalk by endogenous steroid hormones. In certain embodiments the receptors can be an estrogen receptor variant responsive to 4-hydroxytamoxifen or a progesterone-receptor mutant inducible by RU486. In certain embodiments, the nuclear receptor-derived rosiglitazone-responsive transcription switch based on the human nuclear peroxisome proliferator-activated receptor γ(PPARγ) can be used in the regulated expression systems of the present disclosure. In certain embodiments, a variant of steroid-responsive receptors can be the RheoSwitch, that is based on a modified Choristoneura fumiferana ecdysone receptor and the mouse retinoid X receptor (RXR) fused to the Gal4 DNA binding domain and the VP16 trans-activator. In the presence of synthetic ecdysone, the RheoSwitch variant can bind and activate a minimal promoter fused to several repeats of the Gal4-response element.

In certain embodiments, the regulated expression systems disclosed herein can utilize chemically induced dimerization of a DNA-binding protein and a transcriptional activator for the activation of a minimal core promoter fused with a cognate operator. In certain embodiments, the regulated expression systems disclosed herein can utilize the rapamycin-regulated dimerization of FKBP with FRB. In this system the FRB is fused to the p65 trans-activator and FKBP is fused to a zinc finger domain specific for cognate operator sites placed upstream of an engineered minimal interleukin-12 promoter. In certain embodiments, the FKBP can be mutated. In certain embodiments, the regulated expression systems disclosed herein can utilize bacterial gyrase B subunit (GyrB), where GyrB dimerizes in the presence of the antibiotic coumermycin and dissociates with novobiocin.

In certain embodiments, the regulated expression systems of the present disclosure can be used for regulated siRNA expression. In certain embodiments, the regulated siRNA expression system can be a tetracycline, a macrolide, or an OFF- and ON-type QuoRex system. In certain embodiments, the RTI system can utilize a Xenopus terminal oligopyrimidine element (TOP), which blocks translational initiation by forming hairpin structures in the 5′ untranslated region.

In certain embodiments, the regulated expression systems described in the present disclosure can utilize gas-phase controlled expression, e.g., acetaldehyde-induced regulation (AIR) system. The AIR system can employ the Aspergillus nidulans AlcR transcription factor, which specifically activates the PAIR promoter assembled from AlcR-specific operators fused to the minimal human cytomegalovirus promoter in the presence of gaseous or liquid acetaldehyde at nontoxic concentrations.

In certain embodiments, the regulated expression systems of the present disclosure can utilize a Tet-On or a Tet-Off system. In such systems, expression of a one or more GOIs can be regulated by tetracycline or its analogue, doxycycline.

In certain embodiments, the regulated expression system of the present disclosure can utilize a PIP-on or a PIP-off system. In such systems, the expression of proteins can be regulated by, e.g., pristinamycin, tetracycline and/or erythromycin.

### 6. Methods of Producing Recombinant AAV

In certain embodiments of the present disclosure, there is provided a method of producing recombinant AAV (rAAV) comprising: (A) providing a stable mammalian host cell comprising a first exogenous nucleotide sequence integrated at a targeted locus of the genome of the stable mammalian cell, wherein the first exogenous nucleotide sequence comprises two recombination recognition sequences (RRSs) flanking a first selection marker; (B) introducing into the stable mammalian host cell provided in (A) a second exogenous nucleic acid sequence, where the second exogenous nucleic acid sequence comprises two RRSs matching the two RRSs of the first exogenous nucleotide sequence and flanking a sequence encoding one or more of AAV REP, AAV CAP, Ad E2A, Ad E4, Ad E4orf6, Ad E4orf6/7, Ad E2DBP, or Ad VA gene products, e.g., proteins and/or RNA and a second selection marker; (C) introducing a recombinase or a nucleic acid encoding a recombinase, wherein the recombinase recognizes the RRSs; (D) simultaneously with (B) and (C) or sequentially after (B) and (C), introducing, via transposon-mediated genomic integration, a third exogenous nucleic acid sequence encoding one or more of AAV REP, AAV CAP, Ad E2A, Ad E4, Ad E4orf6, Ad E4orf6/7, Ad E2DBP, Ad VA gene products, e.g., proteins and/or RNA, or a polypeptide of interest, and a third selection marker into the genome of the stable mammalian cell; selecting for those stable mammalian host cells stably expressing the second and third selection markers; and culturing the selected stable mammalian host cell under conditions sufficient to produce recombinant AAV.

In certain embodiments, the methods of the present disclosure comprise: A) providing a stable mammalian host cell comprising a first exogenous nucleotide sequence integrated at a targeted locus of the genome of the mammalian host cell, wherein the first exogenous nucleotide sequence comprises a first and a second RRS flanking at least one first selection marker, and a third RRS located between the first and the second RRS, and where all the RRSs are heterospecific; B) introducing into the cell provided in a) a first vector comprising two RRSs matching the first and the third RRS on the at least one integrated exogenous nucleotide sequence and flanking a sequence encoding one or more of AAV REP, AAV CAP, Ad E2A, Ad E4, Ad E4orf6, Ad E4orf6/7, Ad E2DBP, or Ad VA proteins and a selection marker; c) introducing into the cell provided in a) a second vector comprising two RRSs matching the second and the third RRS on the at least one integrated exogenous nucleotide sequence and flanking a sequence encoding one or more of AAV REP, AAV CAP, Ad E2A, Ad E4, Ad E4orf6, Ad E4orf6/7, Ad E2DBP, or Ad VA proteins and a selection marker; D) introducing two or more recombinases, or one or more nucleic acids encoding two or more recombinases, wherein the two or more recombinases recognize the RRSs; and E) simultaneously with B), C), and D), or sequentially after B), C), and D), introducing, via transposon-mediated genomic integration, an additional exogenous nucleic acid sequence encoding one or more of AAV REP, AAV CAP, Ad E2A, Ad E4, Ad E4orf6, Ad E4orf6/7, Ad E2-DNA Binding Protein (E2DBP), Ad VA gene products, e.g., proteins and/or RNA, or a polypeptide of interest, and a selection marker into the genome of the stable mammalian host cell; F) selecting for those mammalian host cells that stably express one or more of the selection markers; and G) culturing the selected mammalian host cell under conditions sufficient to produce recombinant AAV. In certain embodiments, such methods may further comprise recovering the AAV of interest from the cell culture.

In certain embodiments, the stable mammalian host cells selected for expressing the third selection marker comprise one to ten copies of the transposon-mediated genomically integrated third exogenous nucleic acid sequence.

In certain embodiments, the nucleic acid sequence in (D) further comprises a coding sequence encoding a second polypeptide of interest.

In certain embodiments, the first, second and third exogenous nucleotide sequence further comprises an expression cassette of transactivator (rTA) sequence, wherein the rTA sequence is fused to one or more of the first, second and third selection marker. In certain embodiments, the rTA is fused to the first and/or the second selection marker via a 2A peptide. For example, but not by way of limitation, the 2A peptide can be selected from P2A, T2A, E2A, and F2A. In certain embodiments, the rTA is fused to the first and/or the second selection marker via a P2A peptide sequence.

In certain embodiments, the cell culture of the selected stable mammalian host cells is a suspension culture. In certain embodiments, the cell culture of the selected stable mammalian host cells is a 2-dimensional adherent culture. In certain embodiments, the cell culture of the selected stable mammalian host cells is a 3-dimensional matrix culture. In certain embodiments, the cell culture is a serum-free culture.

In certain embodiments, targeted integration of the first exogenous nucleotide sequence is promoted by exogenous nuclease disclosed in the present disclosure in Section 5.2.

In certain embodiments, expression of the one or more proteins is controlled by a regulatable promoter. For example, and not by way of any limitation, the regulatable promoter is a SV40 and/or CMV promoter.

In certain embodiments, a plurality of proteins encoded by the first and/or second exogenous nucleic acid are inducibly expressed. In certain embodiments, expression of the one or more of proteins encoded by the first exogenous nucleotide sequence and/or the second exogenous nucleotide sequence integrated at a targeted locus of the genome of the stable mammalian cell is inducibly expressed.

In certain embodiments, expression of the one or more of proteins encoded by the transposon-mediated genomically integrated third exogenous nucleic acid is inducibly expressed. In certain embodiments, expressions, expression of the one or more of proteins encoded by the transposon-mediated genomically integrated third exogenous nucleic acid is constitutively expressed.

In certain embodiments, one or more of the proteins encoded by the first and/or second exogenous nucleotide sequence integrated at a targeted locus of the genome of the stable mammalian cell is inducibly expressed and one or more of the proteins encoded by the transposon-mediated genomically integrated third exogenous nucleic acid is constitutively expressed. In certain embodiments, one or more of the proteins encoded by the first and/or second exogenous nucleotide sequence integrated at a targeted locus of the genome of the stable mammalian cell is inducibly expressed and one or more of the proteins encoded by the transposon-mediated genomically integrated third exogenous nucleic acid is inducibly expressed. In certain embodiments, one or more of the proteins encoded by the first and/or second exogenous nucleotide sequence integrated at a targeted locus of the genome of the stable mammalian cell is constitutively expressed and one or more of the proteins encoded by the transposon-mediated genomically integrated third exogenous nucleic acid is constitutively expressed. In certain embodiments, one or more of the proteins encoded by first and/or second exogenous nucleotide sequence integrated at a targeted locus of the genome of the stable mammalian cell is constitutively expressed and one or more of the proteins encoded by the transposon-mediated genomically integrated third exogenous nucleic is inducibly expressed.

In certain embodiments, inducibly expressed proteins are induced by the same inducing agent. In certain embodiments, inducibly expressed proteins are induced by two or more inducing agents that are different. Non-limiting examples of such inducing agents include, doxycycline, tetracycline, cumate, isopropyl-B-D-thiogalactopyranoside (IPTG), macrolides, 6-hydroxynicotine, streptogramins, and NADH, or a combination of these.

In certain embodiments, the proteins may be inducibly expressed using regulated expression systems. Non-limiting examples of such inducible promoters for regulated protein expression are Tet-On and Tet-Off systems. In such systems, expression of a one or more GOIs can be regulated by tetracycline or its analogue, doxycycline.

Also provided herein are high-throughput screening methods for reducing the production time for the TI/P producer cells of the present disclosure. In certain embodiments, such high-throughput screening methods comprise:
(A) integrating into a TI host cell, an exogenous nucleotide sequence encoding the Rep and Helper genes to obtain a first plurality of TI pre-packaging clones via single cell cloning;
(B) transducing clones in the first plurality with an rAAV encoding a gene of interest (GOI);
(C) screening the transduced clones for viral genome replication to obtain a second plurality of TI pre-packaging clones exhibiting desirable viral genome replication efficiency upon induction of the Rep and Helper genes;
(D) performing a transient transfection in clones from the second plurality, using an exogenous nucleotide sequence encoding a Cap gene and a GOI to obtain a plurality of producer cell clones; and
(E) screening the plurality of producer cell clones for rAAV production upon induction of the Cap, Rep, and Helper genes.

In certain embodiments, expression of the one or more of the Rep, Helper and Cap proteins employed in the replication assay and/or the transient transfection assay is inducibly expressed. In certain embodiments, the inducibly expressed protein(s) are induced by the same inducing agent. In certain embodiments, the inducibly expressed protein(s) are induced by distinct inducing agents. Non-limiting examples of inducing agents that find use in connection with the replication and/or transient transfection assays described herein include, but are not limited to, doxycycline, tetracycline, cumate, isopropyl-β-D-thiogalactopyranoside (IPTG), macrolides, 6-hydroxynicotine, streptogramins, and NADH, or a combination two or more of these.

In certain embodiments, the inducibly expressed protein(s) can be inducibly expressed using a regulated expression system. For example, but not by way of limitation, one or more of the Rep, Cap, and Helper genes are inducible by tetracycline.

In certain embodiments, screening the transduced clones for viral genome replication or rAAV production is performed using PCR. In certain embodiments, such screening is performed using droplet digital PCR (ddPCR).

### 7. EXAMPLES

### Comparison of Cell Pools and Single Colony Clones Produced by Sequential and Simultaneous Integration of AAV Genes

This study presents a comparison of AAV titers obtained from sequential (TI/P, FIG. 1A) and simultaneous transfection (TIP, FIG. 1B) integrated stable mammalian host cell pools and isolated single cell clones (SCC) using targeted integration (TI) plasmids and/or piggyBac plasmids.

### 7.1 Knock-in Landing Cassette

A knock-in landing cassette that encoded a GFP gene and a hygromycin-thymidine kinase (HYTK) selection marker, flanked by two LoxP sites, L3 and 2L, an additional LoxP site, Loxfas and a frt site, frt3 was constructed (FIG. 2). The GFP expression cassette is flanked by homologous arms of AAVS1 site in HEK293 cells for targeting, AAVS1-LA (left homologous arm) and AAVS1-RA (right homologous arm), respectively. A BFP expression cassette is placed downstream of the right homologous arm to distinguish random integration.

Guide RNA sequences were designed using the CRISPR Guide RNA Design software (Benchling, CA) and manufactured by Integrated DNA Technologies (IDT, Coralville, IA, USA). The selected gRNA was then complexed with Hifi Crispr-cas9 nuclease at room temperature for 15 minutes. The RNP and the knock-in landing cassette were transfected into ten million HEK293 cells using a Neon™ Transfection System and Neon™ Transfection System 100 μL Kit (Thermo Fisher Scientific, Waltham, MA, USA). Transfected cells were selected in 100 μg/mL hygromycin. Once the transfected pool recovered, genomic DNA was isolated and genomic PCR was used to confirm that the landing cassette was knocked in at the correct site. The KI pool was subjected to SCC by limiting dilution using 384 well plates with a target density of 0.6 cells/well. Plates were cultured for 3 weeks at 37° C., 8% CO2, and 80% humidity. Single cell clones were picked and scaled up for characterization. A HEK293 TI host was identified (see FIG. 2 for rAAV titer comparison with Wild-Type (WT) HEK293) based on whether: 1) the landing cassette was correctly integrated, and 2) the TI host could carry out 2-plasmid RMCE and 3-plasmid RMCE as previously described.

### 7.2 Case Study I—Generating Packaging Cell Line

### Expression Plasmids

Front TI plasmid (FIG. 4A) consists of a Tet inducible promoter driving expression of REP gene followed by an expression cassette of transactivator (rTA). Downstream of that lies an SV40 promoter. All the elements mentioned above are flanked by two Cre recombinase recognition sites (L3, Loxfas). TI back plasmid (Figure B) consists of an ORF of the puromycin-N-acetyltransferase (PAC) gene followed by a Tet inducible promoter driving expression of helper genes. All the elements mentioned above are flanked by two Cre recombinase recognition sites (Loxfas, 2L).

PB plasmid (FIG. 4C) consists of a Tet-inducible promoter driving the expression of a CAP gene, followed by a GOI GFP expression cassette flanked by two inverted terminal repeats (ITR). Downstream of that lies a selection marker (SM), for example a blasticidin resistance marker (BSD), a zeocin resistance marker (ZEO), or a hygromycin resistance marker (HYGRO). All the elements mentioned above are flanked by two ITR sites (pITR, different from the ones flanking GFP gene) in the plasmid.

Transfection HEK293 TI host cells were seeded at 4E+5 cells/mL 2 days prior to the transfection. Two different transfection schedules were tested: simultaneous transfection of TI and PB plasmids into HEK293 TI host (TIP strategy); transfection of TI plasmids into HEK293 TI host followed by a transfection of PB plasmids after the TI RMCE pool recovers (TI/P strategy).

For TIP transfections, equal molar of TI front and back plasmids and Piggybac plasmid #1, along with a Cre recombinase and a transposase expression plasmid were used. 30E6 cells were used in each transfection, which was performed by electroporation using MaxCyte. Pools were transferred to selection media 48 hours post transfection.

For TI/P transfections, equal molar of TI front and back plasmids, along with a Cre recombinase plasmid were used. 30e6 cells were used in each transfection, which was performed by electroporation using MaxCyte. Pools were transferred to selection media 48 hours post transfection. Once the RMCE pool recovered, PiggyBac plasmid #1 and a transposase plasmid were transfected by electroporation using MaxCyte into the RMCE pool cells. Pools were transferred to selection media 48 hours post transfection.

### Production and Assays

A 3-day fed-batch shake flask production assay was used to determine the viral genome (VG) and viral particle (VP) productivity of the transfected pools. Cells were seeded on day 0 at 2E6 cells/ml in a commercial production media in shake flasks. Doxcyclin was added to induce the viral gene components on day 0. Batch feed was added on day 1. Cells were shaken at 150 rpm, 37° C. and 8% CO2 throughout the 3-day duration. Cell pellet samples from day 3 were harvested and processed. VG measurement was done by GFP specific droplet PCR and VP measurement by Gyrolab AAVX titer kit. Table 1 summarizes data obtained from the Case Study I.

TABLE 1
Quantitation of TI/P and TIP pools and clones

| | TI/P | | | |
|---|---|---|---|---|
| | cell pools | TI/P cell clones | | |
| Parameter | — | Clone A | Clone B | |
| Titer¹ (10E8 VG/mL) | 3.5 | 10.3 | 8.2 | |
| Total Capsid² (10E8 VP/mL) | 12 | 87 | 27.6 | |
| Full/empty ratio (%) | 26 | 11.7 | 29.5 | |
| | TIP | | | |
| | cell pools | TIP cell clones | | |
| Parameter | — | Clone C | Clone D | Clone E |
| Titer¹ (10E8 VG/mL) | 0.4x | 4.7 | 1.7 | 4.5 |
| Total Capsid² (10E8 VP/mL) | 31 | 664 | 79.4 | 176 |
| Full/empty ratio (%) | 1.3 | 0.7 | 2.2 | 2.5 |

¹ddPCR (Droplet digital polymerase chain reaction);
²Gyrolab ® AAVX Titer

### 7.3 Case Study II

### Expression Plasmids

Front and Back TI plasmids are as described in Case Study I (FIGS. 4A-4B). PiggyBac plasmid #1 is as described in Case Study I (FIG. 4C). PiggyBac plasmid #2 (FIG. 4D) consists of a Tet-inducible promoter driving the expression of a REP gene, followed by the PAC gene. All the elements mentioned above are flanked by two pITR sites in the plasmid. PiggyBac plasmid #3 (FIG. 4E) consists of a Tet-inducible promoter driving the expression of helper genes, followed by the expression of a zeocin resistance gene (ZEO). All the elements mentioned above are flanked by two pITR sites in the plasmid.

### Transfection

For HEK293 packaging cell line, HEK293 TI host cells were seeded at 4E+5 cells/mL 2 days prior to the transfection. Equal molar of TI front and back plasmids, along with a Cre recombinase plasmid were used. 30E+6 cells were used in each transfection, which was performed by electroporation using MaxCyte. Pools were transferred to selection media 48 hours post transfection. Once the RMCE pool recovered, equal molar of PiggyBac plasmid #1, #2, and #3, along with a transposase plasmid, were transfected by electroporation using MaxCyte into the RMCE pool cells. Pools were transferred to selection media 48 hours post transfection. For H10 packaging cell line, H10 TI host cells were seeded at 4E+5 cells/ml 2 days prior to the transfection. On the day of transfection, 30E+6 H10 cells were transfected with 12.5 μg of TI front plasmid, 12.5 μg of TI back plasmid and 5 μg of Cre recombinase plasmid were complexed with 60 μL of PEIpro. Pools were transferred to selection media 48 hours post transfection.

### Selection

The TI pool was initially selected at 1.5 μg/mL of puromycin and 0.5 μM of 1-(2-deoxy-2-fluoro-1-D-arabinofuranosyl)-5-iodouracil (FIAU) at 6E+5 cells/mL. TI packaging pools were recovered when viability was >90%.

### Production and Assays

Production and assay procedures are as described in Case study I.

TABLE 2
Comparison of TI/P Pools vs PiggyBac alone Pool

| | | Titer (ddPCR) | Full/empty |
|---|---|---|---|
| | Method | 10E8 VG/mL | ratio (%) |
| Case I | TI/P | 109 | 2.8 |
| | PiggyBac alone | 56 | 0.6 |
| | Fold increase | 1.9x | 4.6x |
| Case II | TI/P | 36 | ~100 |
| | PiggyBac alone | 28 | 53.7 |
| | Fold increase | 1.3x | 1.8x |

### 7.4 Case Study III—Generating TI/P Producer Cell Line

### Expression Plasmids

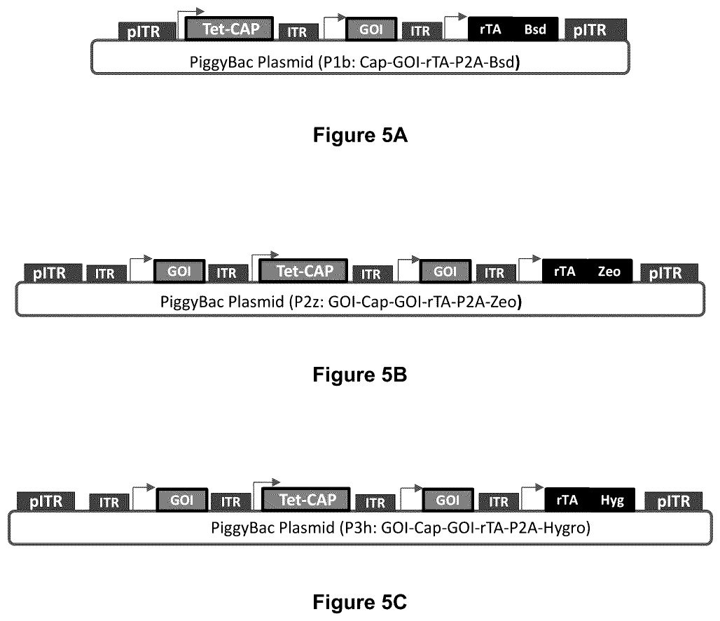

FIGS. 5A-5C illustrate exemplary combinations of CAP and GOI-encoding PiggyBac (PB) plasmids that can be transfected into a TI packaging host to make producer cell lines. The PiggyBac (PB) transposon donor plasmids comprise a Tet-inducible promoter driving expression of a CAP gene, followed by a GFP expression cassette flanked by two AAV inverted terminal repeats (ITR). Downstream to the Tet-driven CAP gene is a GOI expression cassette flanked by two AAV inverted terminal repeats (ITR) (FIG. 5A). Downstream to the ITR is a EF1a promoter for driving expression of transactivator (rTA) followed by a selection marker (SM), for example a blasticidin resistance marker (BSD), a zeocin resistance marker (ZEO), or a hygromycin resistance marker (HYGRO). The selection marker in the PB plasmid can optionally be fused to an expression cassette of transactivator (rTA) element via a sequence encoding a 2A peptide sequence (e.g., P2A, T2A, E2A, F2A). Some PB transposon donor plasmids comprise additionally, a second copy of a GOI sequence (e.g., GFP) flanked by two AAV ITRs upstream to the Tet-driven CAP gene (FIGS. 5B, 5C). All the elements mentioned above are flanked by two PB ITR sites (different from those flanking GOI).

### Transfection

For TI/P transfections, TI pre-packaging cells were seeded at 2E+6 cells/ml 1-3 hours prior to the transfection. 46E+6 cells were transfected with PB transposase plasmid (3 μg) or PB transposase mRNA (13 μg), and PB transposon donor plasmid (27 μg) using polyethylenimine (PEI).

### Selection

Two days post transfection, the transfected cell pools were transferred to selection media. Inclusion of blasticidin, zeocin and hygromycin in the selective media is based on the combination of PB transposon donor plasmids used in transfection. TI/P producer pools were recovered when viability was >90%.

### Production and Assays

A 5-day fed-batch shake flask production assay was used to determine productivity of transfected pools. Cells were seeded on day 0 at 20E+6 cells/ml (17 ml) in 125 ml shake flasks. Dox was added to activate expression of viral genes and batch feed was added on day 1 and day 4. Cells were cultured with shaking at 150 rpm, at 37° C. and 8% CO2 throughout the 5-day run. Cell samples from day 5 were collected and lysed for titer measurement using a GFP specific droplet PCR and capsid measurement kit (Gyrolab AAVX titer kit). A ViCell XR cell counter was for all steps.

### Results

Table 3 summarizes the results of TI/P pool titers from TI packaging cells transfected with PiggyBac plasmids comprising Cap and GOI sequences.

TABLE 3
Effect of selection markers on TI/P titers and Cap/GOI copy numbers

| | | | | | | | |
|---|---|---|---|---|---|---|---|
| BSD Selection | + | − | − | + | + | − | + |
| ZEO Selection | − | + | − | + | − | + | + |
| HYGRO | − | − | + | − | + | + | + |
| Selection | | | | | | | |
| Sample ID → | P1b | P2z | P3h | P1b2z | P1b3h | P2z3h | P1b2z3h |
| Assay ↓ | | | | | | | |
| ddPCR titer | 1.84E+10 | 3.20E+10 | 3.80E+10 | 5.60E+10 | 1.30E+11 | 1.30E+11 | 1.20E+11 |
| (vg/ml) | | | | | | | |
| GOI copy | 3.8 | 2.5 | 1.8 | 7.5 | 9.1 | 7 | 11.1 |
| number per cell | | | | | | | |
| Cap copy | 3.8 | 1.2 | 1.1 | 4.5 | 5.3 | 5 | 9.2 |
| number per cell | | | | | | | |

### 7.5 High-Throughput Process for TI/P Producer Cell Production

### 7.5.1 Rapid Screening

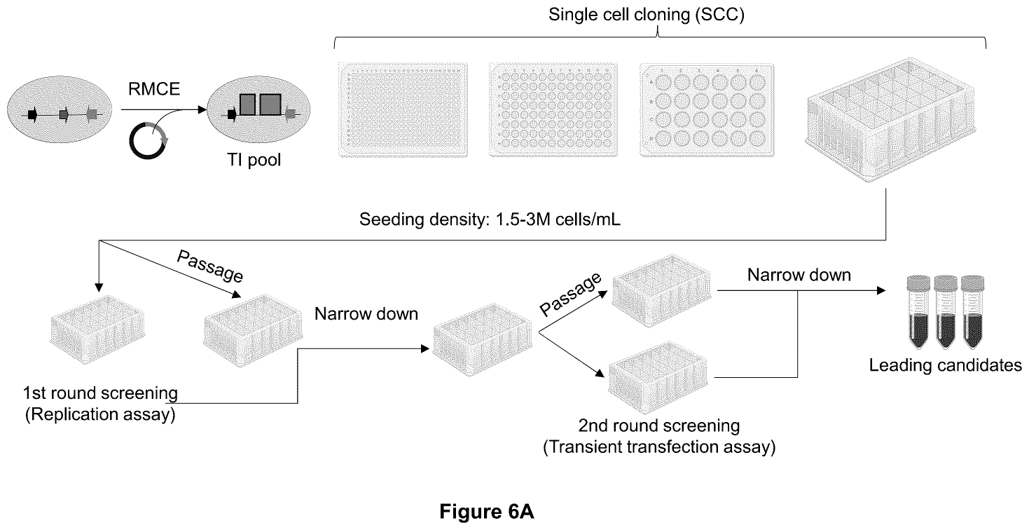

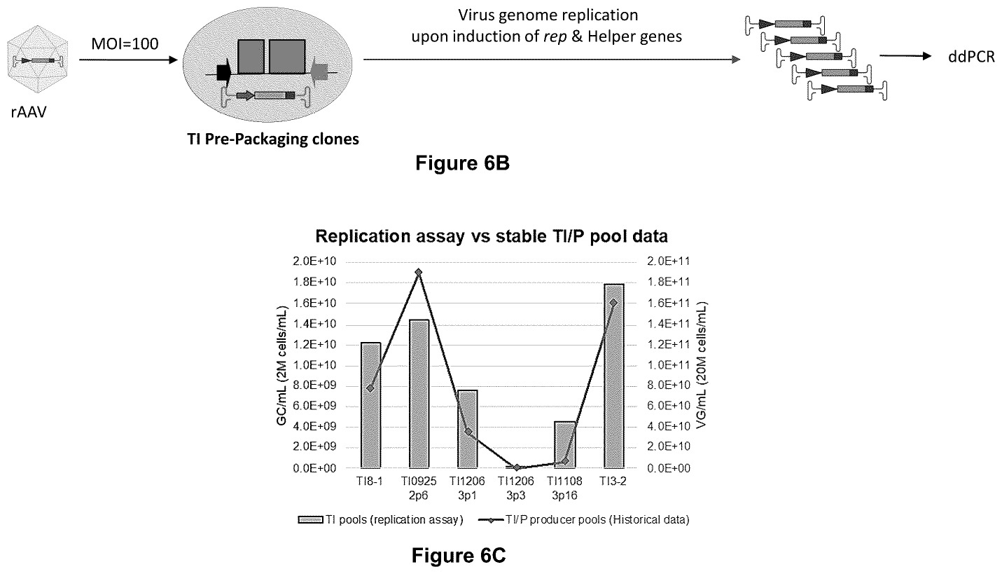

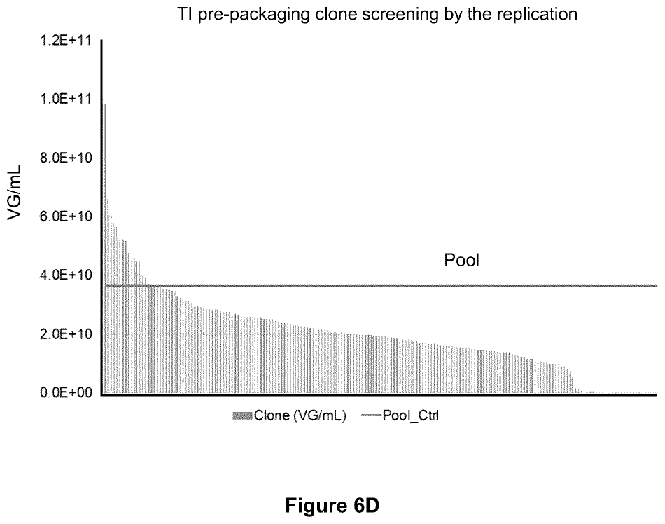

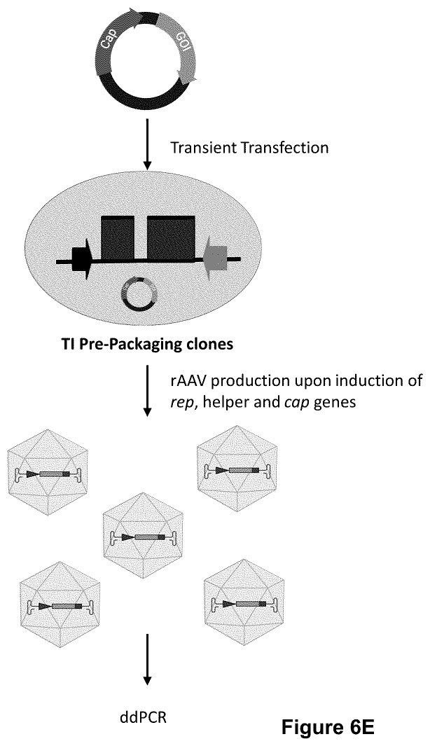

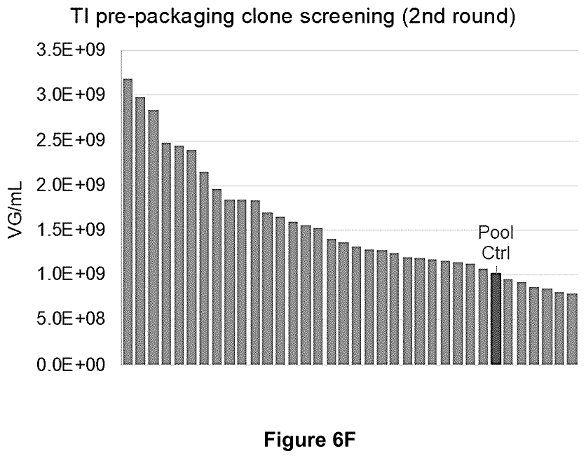

A rapid screening method was developed that significantly reduced production of TI pre-packaging host cells and TI/P producer cells (FIG. 6A). Briefly, HEK293 TI host cells were seeded at 4E+5 cells/mL 2 days prior to the transfection and transfected with a plasmid comprising the Rep and Helper sequences under the control of a Tet promoter. Post transfection and RMCE pool recovery, clones generated via single cell cloning were seeded and cultured in multiwell culture plates at a density of 1.5-3×10⁶ cells/ml for the first round of screening (“Replication Assay”) to evaluate each clone's replication capability. As illustrated in FIG. 6B, the clones from each well were transduced with rAAV carrying the gene of interest (MOI=100) and viral genome replication following induction of the Rep and Helper genes was measured by droplet digital PCR, ddPCR. As illustrated in FIG. 6A, clones selected based on the first round of screening were taken through a second round of screening (“Transient Transfection Assay”). As illustrated in FIG. 6E, clones selected based on the replication assay were transiently transfected with a vector that comprises the Cap and GOI sequences and the rAAV particles produced post induction of the Rep, Helper and Cap genes measured by ddPCR.

### Results

### Replication Assay

FIGS. 6C and 6D shows a comparison of the results of the replication assay versus stable TI/P producer pool data. The replication assay allows evaluation of replication capability of the TI test clones in about 4 days. 190 clones were screened using the replication assay using both VG/mL and VG/cell as selection criteria. 36 clones identified by screening advanced to the second round of screening (FIGS. 6E-6F).

### Transient Transfection Assay

### Results:

FIGS. 6E and 6F show the results of the second round of screening using the transfection assay, which can also be completed in about 4 days. Of the 36 clones identified 80% performed better than the stable TI prepackaging pool. Candidate clones identified in the replication assay also performed well in the transfection assay as demonstrated by at least a 3-fold increase in VG titer. In conclusion, the high-throughput assay that is completed in about 8 days has significant advantages over traditional methods that take 4-5 weeks to complete.

7.5.2 Production of TI/P producer pools

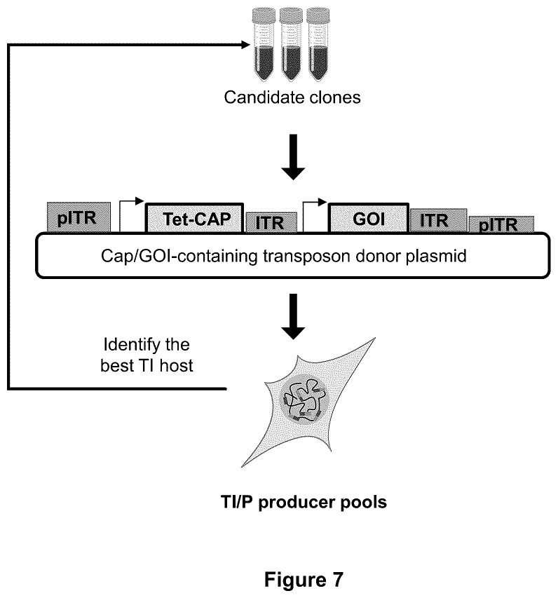

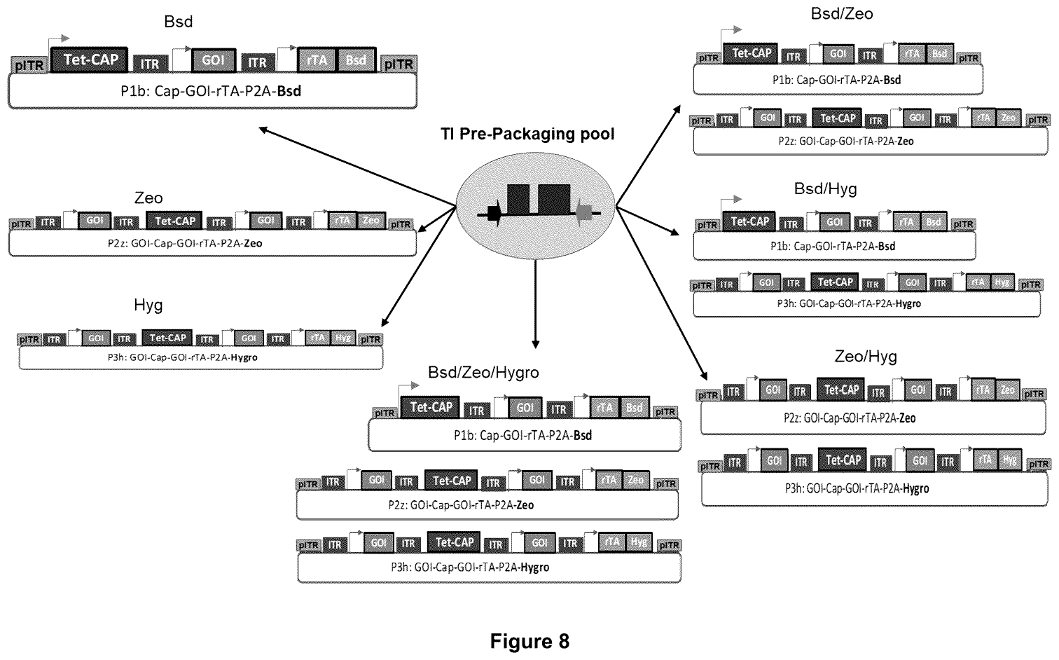

Clones that showed high performance in the replication and transient transfection assays were tested for insertion of the Cap/GOI elements using the PiggyBac transposase (FIG. 7). Productivity of TI/P clone pools generated from each TI host was evaluated for various combinations of double and triple selection markers (FIG. 8) using protein expression, integrated copy number and rAAV titer as readouts.

### Results:

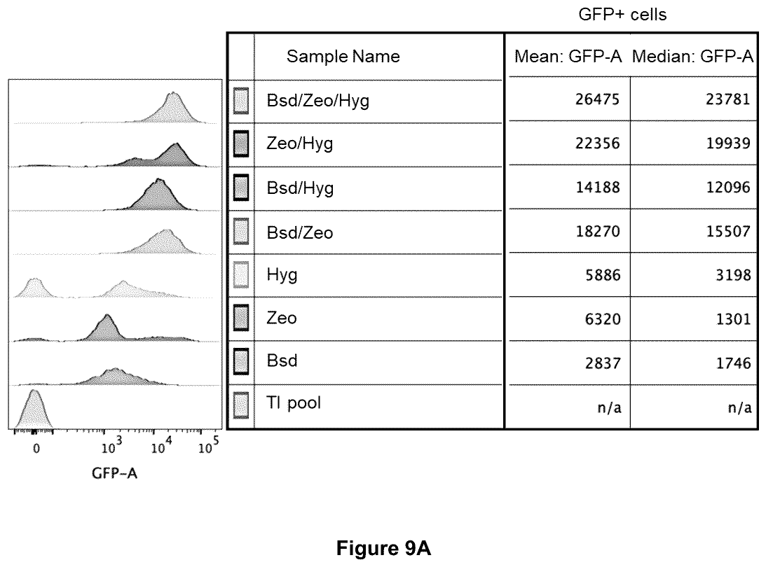

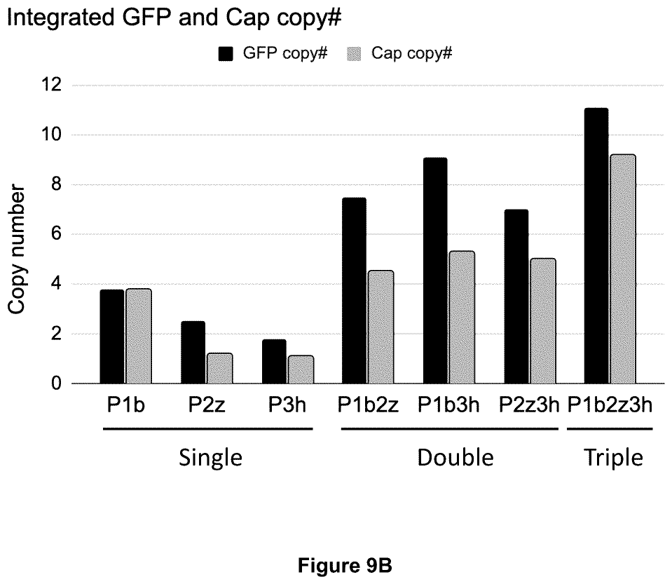

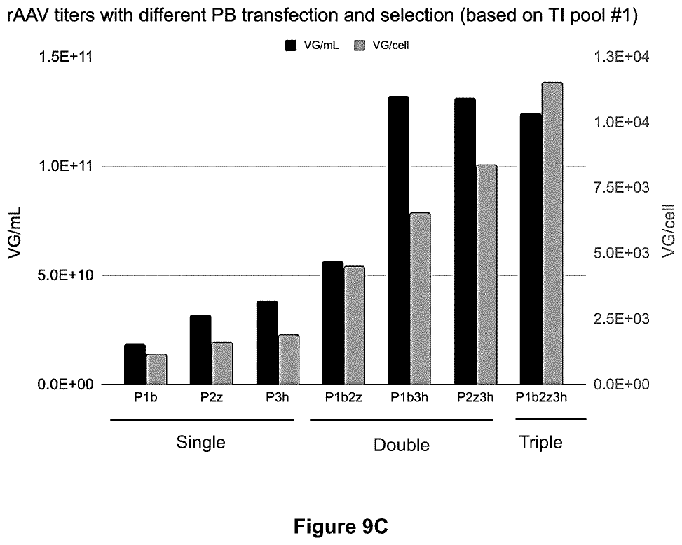

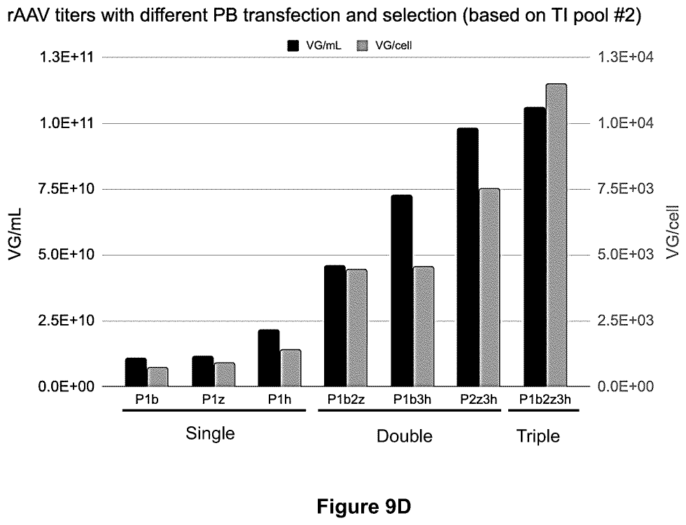

Double and triple selection was found to yield cells with high % GFP expression and high mean fluorescence intensity (FIG. 9A), higher integrated copies of Cap/GOI (FIG. 9B), and higher rAAV titers (FIG. 9C), when seeded at 20×10⁶/ml, induced on day 1 and 4 and harvested on day 5. A similar trend was observed using a different TI pool for TI/P producer pool generation (FIG. 9D).

### 7.6 Comparison of Viral Titer Between TI/P Producer Clonal Host and Triple Transient Transfection

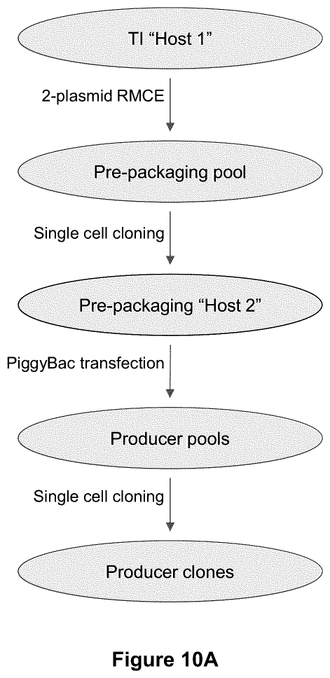

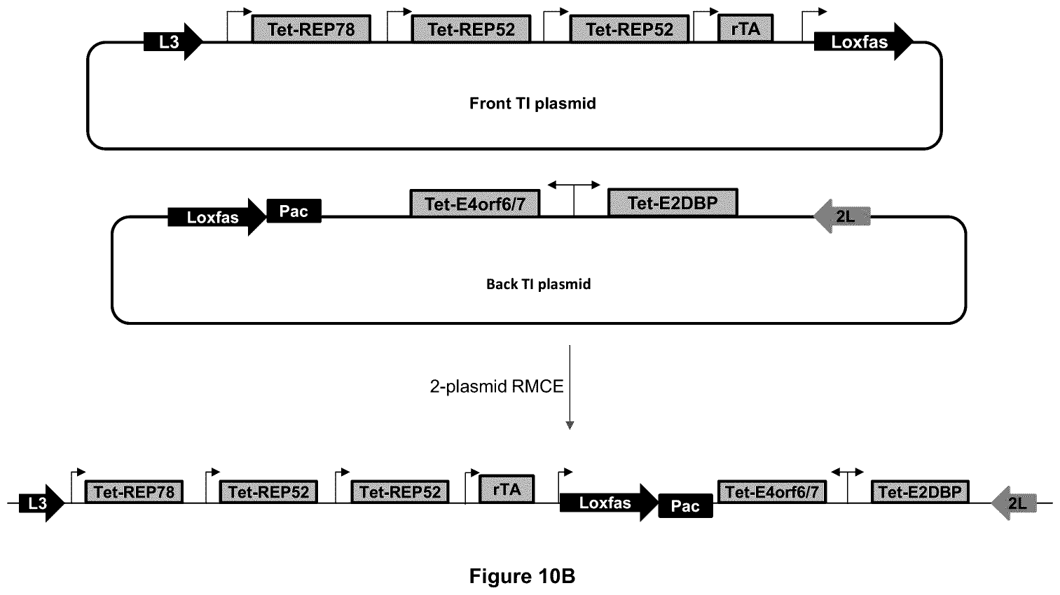

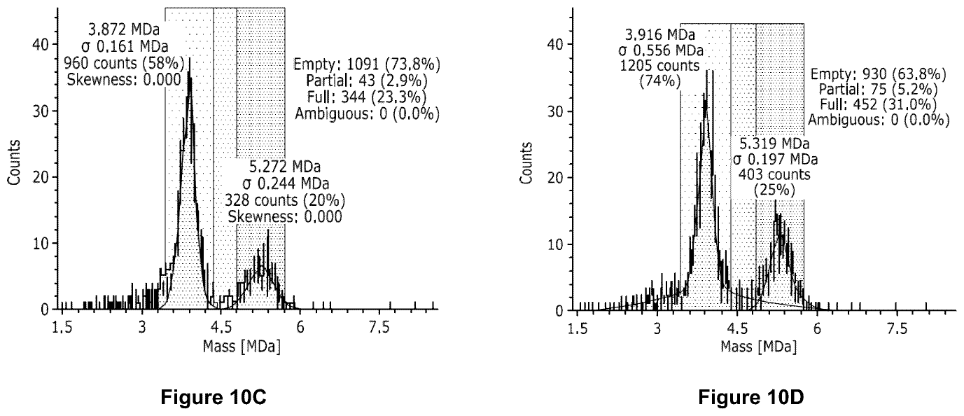

This study presents a comparison of AAV titers obtained by methods employing TI/P producer clones (FIGS. 10A-10B) and triple transient transfection.

Two TI plasmids (front TI plasmid and back TI plasmid) were transfected into the TI host (Host 1) as described above using 2-plasmid RMCE to generate a pre-packaging pool containing REP and Helper genes. Once recovered, the pre-packaging pool was subjected to single cell cloning to obtain a pre-packaging host (Host 2). Producer pools were then generated by stably transfecting the GOI and Cap gene into the pre-packaging host using a PiggyBac transposase. Once recovered, the producer pools were subjected to single cell cloning to obtain producer clones.

### Evaluation of Viral and Capsid Titer

Clones were picked and scaled up for shake flask production. Viral titer (VG/mL) was measured by GFP specific droplet PCR and capsid titer was (VP/mL) measured by AAVX-based titer assay. Crude lysate full/empty ratio (%) was determined by division of the VG/mL by VP/mL. Full/empty ratio (%) of rAAV purified from crude lysate generated from top TI/P producer clone and transient transfection was confirmed by mass photometry (MP). MP-determined Full/empty ratio (%) was calculated by defining regions of full, partial and empty particles in the mass histograms based on the empty particle calibrant mass and the transgene size. The data presented in Table 4 and FIGS. 10C-10D indicate that top TI/P producer clone has a higher VG/mL and slightly lower full/empty ratio (%) compared to standard triple transient transfection method when using the same sized GOI.

TABLE 4
Comparison of viral titers using TI/P producer clones and
triple transient transfection method

| | | | Crude | MP- |
|---|---|---|---|---|
| | | | lysate | determined |
| | | | Full/empty | Full/empty |
| | VG/mL | VP/mL | ratio (%) | ratio (%) |
| Host 2 clone | 4.0E11 | 1.2E12 | 40.0 | 23.3 |
| Triple plasmid transient | 5.3E10 | 1.5E11 | 36.3 | 31.0 |
| transfection | | | | |

1. A stable mammalian host cell comprising:
A) a first exogenous nucleic acid sequence integrated at a targeted locus of the genome of the stable mammalian host cell, where the first exogenous nucleic acid sequence encodes:
(i) one or more of adeno-associated virus (AAV) REP, AAV CAP, adenovirus (Ad) E2A, Ad E4, Ad E4orf6, Ad E4orf6/7, Ad E2-DNA Binding Protein (E2DBP), or Ad VA gene products; and
(ii) a first selection marker;
and comprises two recombination recognition sequences (RRSs) flanking (A) (i) and A (ii); and
B) a second exogenous nucleic acid sequence integrated at least once in the genome of the stable mammalian cell comprising inverted terminal repeats (ITRs) flanking coding sequences for:
(i) a polypeptide of interest (POI);
(ii) one or more of AAV REP, AAV CAP, Ad E2A, Ad E4, Ad E4orf6, Ad E4orf6/7, Ad E2DBP, or Ad VA gene products; and
(iii) a second selection marker.

2. The stable mammalian host cell of claim 1, wherein:
(a) the first exogenous nucleic acid sequence encodes AAV REP, Ad E2A, Ad E4, Ad E4orf6, Ad E4orf6/7, Ad E2DBP, and Ad VA gene products; and/or
(b) the second exogenous nucleic acid encodes AAV CAP.

3. The stable mammalian host cell of claim 1, comprising one to ten additional exogenous nucleic acids comprising inverted terminal repeats flanking coding sequences for:
A) one or more of AAV REP, AAV CAP, Ad E2A, Ad E4, Ad E4orf6, Ad E4orf6/7, Ad E2DBP, or Ad VA gene products; and
B) a selection marker; and
wherein the one to ten additional exogenous nucleic acids are each integrated at least once in the genome of the stable mammalian cell.

4. The stable mammalian host cell of claim 3, wherein the one or more of the one to ten additional exogenous nucleic acids further comprise a coding sequence for a polypeptide of interest (POI) within the flanking inverted terminal repeats.

5. The stable mammalian host cell of claim 1, further comprising a coding sequence for a second polypeptide of interest within the flanking inverted terminal repeats.

6. The stable mammalian host cell of claim 1, further comprising an expression cassette of transactivator (rTA) sequence, wherein the rTA sequence is fused to the first and/or the second selection marker.

7. The stable human cell of claim 1, wherein the stable mammalian host cell is a HEK293 cell.

8. The stable mammalian host cell of claim 1, wherein:
(a) one or more of the proteins encoded by the first exogenous nucleic acid sequence is inducibly expressed and one or more of the proteins encoded by the second exogenous nucleic acid sequence is constitutively expressed;
(b) one or more of the proteins encoded by the first exogenous nucleic acid sequence is inducibly expressed and one or more of the proteins encoded by the second exogenous nucleic acid sequence is inducibly expressed;
(c) one or more of the proteins encoded by the first exogenous nucleic acid sequence is constitutively expressed and one or more of the proteins encoded by the second exogenous nucleic acid sequence is constitutively expressed; or
(d) one or more of the proteins encoded by first exogenous nucleic acid sequence is constitutively expressed and one or more of the proteins encoded by the second exogenous nucleic acid sequence is inducibly expressed.

9. A method of producing recombinant AAV comprising:
A) providing a stable mammalian host cell comprising a first exogenous nucleotide sequence integrated at a targeted locus of the genome of the stable mammalian cell, wherein the first exogenous nucleotide sequence comprises two recombination recognition sequences (RRSs) flanking a first selection marker;
B) introducing into the stable mammalian host cell provided in (A) a second exogenous nucleic acid sequence, where the second exogenous nucleic acid sequence comprises two RRSs matching the two RRSs of the first exogenous nucleotide sequence and flanking a sequence encoding one or more of AAV REP, AAV CAP, Ad E2A, Ad E4, Ad E4orf6, Ad E4orf6/7, Ad E2DBP, or Ad VA gene products and a second selection marker;
C) introducing a recombinase or a nucleic acid encoding a recombinase, wherein the recombinase recognizes the RRSs;
D) simultaneously with B) and C) or sequentially after B) and C), introducing, via transposon-mediated genomic integration, a third exogenous nucleic acid sequence encoding one or more of AAV REP, AAV CAP, Ad E2A, Ad E4, Ad E4orf6, Ad E4orf6/7, Ad E2DBP, Ad VA gene products, or a polypeptide of interest, and a third selection marker into the genome of the stable mammalian cell;
E) selecting for those stable mammalian host cells stably expressing the second and third selection markers; and
F) culturing the selected stable mammalian host cell under conditions sufficient to produce recombinant AAV.

10. The method of claim 9, further comprising recovering the AAV of interest from the cell culture.

11. The method of claim 9, wherein the nucleic acid sequence in step (D) further comprises a coding sequence encoding a second polypeptide of interest.

12. The method of claim 9, wherein the first, second and third exogenous nucleotide sequence further comprises an expression cassette of transactivator (rTA) sequence, wherein the rTA sequence is fused to one or more of the first, second and third selection marker.

13. The method of claim 9, wherein the stable mammalian host cell is a HEK293 cell.

14. The method of claim 9, wherein targeted integration of the first exogenous nucleotide sequence is promoted by an exogenous nuclease.

15. The method of claim 9, wherein:
(a) one or more of the proteins encoded by the first and/or second exogenous nucleotide sequence integrated at a targeted locus of the genome of the stable mammalian cell is inducibly expressed and one or more of the proteins encoded by the transposon-mediated genomically integrated third exogenous nucleic acid is constitutively expressed;
(b) one or more of the proteins encoded by the first and/or second exogenous nucleotide sequence integrated at a targeted locus of the genome of the stable mammalian cell is inducibly expressed and one or more of the proteins encoded by the transposon-mediated genomically integrated third exogenous nucleic acid is inducibly expressed;
(c) one or more of the proteins encoded by the first and/or second exogenous nucleotide sequence integrated at a targeted locus of the genome of the stable mammalian cell is constitutively expressed and one or more of the proteins encoded by the transposon-mediated genomically integrated third exogenous nucleic acid is constitutively expressed; or
(d) one or more of the proteins encoded by first and/or second exogenous nucleotide sequence integrated at a targeted locus of the genome of the stable mammalian cell is constitutively expressed and one or more of the proteins encoded by the transposon-mediated genomically integrated third exogenous nucleic is inducibly expressed.

16. The method of claim 9, wherein the stable mammalian host cells selected for expressing the third selection marker comprise one to ten copies of the transposon-mediated genomically integrated third exogenous nucleic acid sequence.

17. A method of screening rAAV producer cell clones comprising:
(a) integrating into a TI host cell, an exogenous nucleotide sequence encoding Rep and Helper genes to obtain a first plurality of TI pre-packaging clones via single cell cloning;
(b) transducing clones in the first plurality with an rAAV encoding a gene of interest (GOI);
(c) screening the transduced clones for viral genome replication to obtain a second plurality of TI pre-packaging clones exhibiting desirable viral genome replication efficiency upon induction of the Rep and Helper genes;
(d) performing a transient transfection in clones from the second plurality, using an exogenous nucleotide sequence encoding a Cap gene and a GOI to obtain a plurality of producer cell clones; and
(e) screening the plurality of producer cell clones for rAAV production upon induction of the Cap, Rep, and Helper genes.

18. The method of claim 17, wherein the TI host cell is a HEK293 cell.

19. The method of claim 17, wherein the integration of the exogenous nucleotide sequence encoding Rep and Helper genes is promoted by an exogenous nuclease.

20. The method of claim 17, wherein:
(a) one or more of the proteins encoded by the exogenous nucleotide sequence encoding Rep and Helper genes is inducibly expressed and one or more of the proteins encoded by the exogenous nucleotide sequence encoding a Cap gene and a GOI is constitutively expressed;
(b) one or more of the proteins encoded by the exogenous nucleotide sequence encoding Rep and Helper genes is inducibly expressed and one or more of the proteins encoded by the exogenous nucleotide sequence encoding a Cap gene and a GOI is inducibly expressed;
(c) one or more of the proteins encoded by the exogenous nucleotide sequence encoding Rep and Helper genes is constitutively expressed and one or more of the proteins encoded by the exogenous nucleotide sequence encoding a Cap gene and a GOI is constitutively expressed; or
(d) one or more of the proteins encoded by the exogenous nucleotide sequence encoding Rep and Helper genes is constitutively expressed and one or more of the proteins encoded by the exogenous nucleotide sequence encoding a Cap gene and a GOI is inducibly expressed.
